(19) United States
(12) Patent Application Publication
(10) Pub. No.: US 2006/0247269 A1
(43) Pub. Date: Nov. 2, 2006

(54) Thienopyridone derivatives as kinase inhibitors

(76) Inventors: Daniel Christopher Brookings, Reading (GB); Jeremy Martin Davis, Wokingham (GB); Barry John Langham, Reading (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/561,050

(22) PCT Filed: Jun. 18, 2004
(86) PCT No.: PCT/GB04/02644

§ 371 Date: Jun. 29, 2006

(30) Foreign Application Priority Data
Jun. 20, 2003 (GB) 0314490.4
Dec. 19, 2003 (JP) 0329495.6

(51) Int. Cl. A61K 31/4743; C07D 498/02

(52) U.S. Cl. 514/301; 546/114

(57) ABSTRACT

A series of thieno[2,3-b]pyridin-6(7H)-one derivatives, substituted in the 2-position by a carbonyl- or sulfonyl-linked pyrrolidin-3-yl, pyrrolidin-3-ylamino or related moiety, being inhibitors of p38 MAP kinase, are accordingly of use in medicine, for example in the treatment and/or prevention of immune or inflammatory disorders.

This invention relates to a series of thienopyridone derivatives, to compositions containing them, to processes for their preparation and to their use in medicine.

Immune and inflammatory responses involve a variety of cell types with control and co-ordination of the various interactions occurring via both cell-cell contacts (e.g. integrin interactions with their receptors) and by way of intercellular signalling molecules. A large number of different signalling molecules are involved, including cytokines, lymphocytes, chemokines and growth factors.

Cells respond to such intercellular signalling molecules by means of intracellular signalling mechanisms that include protein kinases, phosphatases and phospholipases. There are five classes of protein kinase of which the major ones are the tyrosine kinases and the serine/threonine kinases [Hunter, T., Methods in Enzymology (Protein Kinase Classification), p. 3, Hunter, T. and Sefton, B. M. eds. vol. 200, Academic Press, San Diego, 1991].

One sub-class of serine/threonine kinases is the mitogen activated protein (MAP) kinases of which there are at least three families which differ in the sequence and size of the activation loop [Adams, J. L. et al., Progress in Medicinal Chemistry pp. 1-60, King, F. D. and Oxford, A. W. eds., vol. 38, Elsevier Science, 2001]: (i) the extracellular regulated kinases (ERKs); (ii) the c-Jun NH₂ terminal kinases or stress activated kinases (JNKs or SAP kinases); and (iii) the p38 kinases which have a threonine-glycine-tyrosine (TGY) activation motif. Both the JNKs and p38 MAP kinases (p38 MAPKs) are primarily activated by stress stimuli including, but not limited to, proinflammatory cytokines, e.g. tumour necrosis factor (TNF) and interleukin-1 (IL-1), ultraviolet light, endotoxin and chemical or osmotic shock.

Four isoforms of p38 MAPK have been described (p38α/β/γ/δ). The human p38α enzyme was initially identified as a target of cytokine-suppressive anti-inflammatory drugs (CSAIDs) and the two isoenzymes found were initially termed CSAID binding protein-1 (CSBP-1) and CSBP-2 [Lee, J. C. et al., Nature (London), 1994, 372, 739-46]. CSBP-2 is now widely referred to as p38α and differs from CSBP-1 in an internal sequence of 25 amino acids as a result of differential splicing of two exons that are conserved in both mouse and human [McDonnell, P. C. et al., Genomics, 1995, 29, 301-2]. CSBP-1 and p38α are expressed ubiquitously and there is no difference between the two isoforms with respect to tissue distribution, activation profile, substrate preference or CSAID binding. A second isoform is p38β which has 70% identity with p38α. A second form of p38β termed p38β2 is also known and of the two this is believed to be the major form. p38α and p38β2 are expressed in many different tissues. However in monocytes and macrophages p38α is the predominant kinase activity [Lee, J. C., ibid; Jing, Y. et al., J. Biol. Chem., 1996, 271, 10531-34; Hale, K. K. et al., J. Immun., 1999, 162, 4246-52]. p38γ and p38δ (also termed SAP kinase-3 and SAP kinase-4 respectively) have ˜63% and ˜61% homology to p38α respectively. p38γ is predominantly expressed in skeletal muscle whilst p38δ is found in testes, pancreas, prostate, small intestine and in certain endocrine tissues.

All p38 homologues and splice variants contain a 12 amino acid activation loop that includes a Thr-Gly-Tyr (TGY) motif. Dual phosphorylation of both Thr-180 and Tyr-182 in the TGY motif by a dual specificity upstream kinase is essential for the activation of p38 and results in a >1000-fold increase in specific activity of these enzymes [Doza, Y. N. et al., FEBS Left., 1995, 364, 7095-8012]. This dual phosphorylation is effected by MKK6 and under certain conditions the related enzyme MKK3 [Enslen, H. et al., J. Biol. Chem., 1998, 273, 1741-48]. MKK3 and MKK6 belong to a family of enzymes termed MAPKK (mitogen activated protein kinase kinase) which are in turn activated by MAPKKK (mitogen activated kinase kinase kinase) otherwise known as MAP3K.

Several MAP3Ks have been identified that are activated by a wide variety of stimuli including environmental stress, inflammatory cytokines and other factors. MEKK4/MTK1 (MAP or ERK kinase kinase/MAP three kinase-1), ASK1 (apoptosis stimulated kinase) and TAK1 (TGF-β-activated kinase) are some of the enzymes identified as upstream activators of MAPKKs. MEKK4/MTK1 is thought to be activated by several GADD-45-like genes that are induced in response to environmental stimuli and which eventually lead to p38 MAPK activation [Takekawa, M. and Saito, H., Cell, 1998, 95, 521-30]. TAK1 has been shown to activate MKK6 in response to transforming growth factor-β (TGF-β). TNF-stimulated activation of p38 MAPK is believed to be mediated by the recruitment of TRAF2 [TNF receptor associated factor] and the Fas adaptor protein, Daxx, which results in the activation of ASK1 and subsequently p38 MAPK.

Several substrates of p38 MAPK have been identified including other kinases [e.g. MAPK activated protein kinase 2/3/5 (MAPKAP 2/3/5), p38 MAPK regulated/activated protein kinase (PRAK), MAP kinase-interacting kinase 1/2 (MNK1/2), mitogen- and stress-activated protein kinase 1 (MSK1/RLPK) and ribosomal S6 kinase-B (RSK-B)]; transcription factors [e.g. activating transcription factor 2/6 (ATF2/6), monocyte-enhancer factor-2A/C (MEF2A/C), C/EBP homologous protein (CHOP), Elk1 and Sap-1a1]; and other substrates [e.g. cPLA2, p47phox].

MAPKAP K2 is activated by p38 MAPK in response to environmental stress. Mice engineered to lack MAPKAP K2 do not produce TNF in response to lipopolysaccharide (LPS). Production of several other cytokines such as IL-1, IL-6, IFN-g and IL-10 is also partially inhibited [Kotlyarov, A. et al., Nature Cell Biol., 1999, 1, 94-7]. Further, MAPKAP K2 from embryonic stem cells from p38α null mice was not activated in response to stress and these cells did not produce IL-6 in response to IL-1 [Allen, M. et al., J. Exp. Med., 2000, 191, 859-69]. These results indicate that MAPKAP K2 is not only essential for TNF and IL-1 production but also for signalling induced by cytokines. In addition MAPKAP K2/3 phosphorylate and thus regulate heat shock proteins HSP 25 and HSP 27 which are involved in cytoskeletal reorganization.

Several small molecule inhibitors of p38 MAPK have been reported which inhibit IL-1 and TNF synthesis in human monocytes at concentrations in the low μM range [Lee, J. C. et al., Int. J. Immunopharm., 1988, 10, 835] and exhibit activity in animal models which are refractory to cyclooxygenase inhibitors [Lee, J. C. et al., Annals N.Y. Acad. Sci., 1993, 696, 149]. In addition these small molecule inhibitors are known to decrease the synthesis of a wide variety of pro-inflammatory proteins including IL-6, IL-8, granulocyte/macrophage colony-stimulating factor (GM-CSF) and cyclooxygenase-2 (COX-2). TNF-induced phosphorylation and activation of cytosolic PLA2, TNF-induced expression of VCAM-1 on endothelial cells and IL-1 stimulated synthesis of collagenase and stromelysin are also inhibited by small molecule inhibitors of p38 MAPK [Cohen, P., Trends Cell Biol., 1997, 7, 353-61].

A variety of cells including monocytes and macrophages produce TNF and IL-1. Excessive or unregulated TNF production is implicated in a number of disease states including Crohn's disease, ulcerative colitis, pyresis, rheumatoid arthritis, rheumatoid spondylitis, osteoarthritis, gouty arthritis and other arthritic conditions, toxic shock syndrome, endotoxic shock, sepsis, septic shock, gram negative sepsis, bone resorption diseases, reperfusion injury, graft vs. host reaction, allograft rejection, adult respiratory distress syndrome, chronic pulmonary inflammatory disease, silicosis, pulmonary sarcoidosis, cerebral malaria, scar tissue formation, keloid formation, fever and myalgias due to infection, such as influenza, cachexia secondary to acquired immune deficiency syndrome (AIDS), cachexia secondary to infection or malignancy, AIDS or AIDS related complex.

Excessive or unregulated IL-1 production has been implicated in rheumatoid arthritis, osteoarthritis, traumatic arthritis, rubella arthritis, acute synovitis, psoriatic arthritis, cachexia, Reiter's syndrome, endotoxemia, toxic shock syndrome, tuberculosis, atherosclerosis, muscle degeneration, and other acute or chronic inflammatory diseases such as the inflammatory reaction induced by endotoxin or inflammatory bowel disease. In addition IL-1 has been linked to diabetes and pancreatic β cell destruction [Dinarello, C. A., J. Clinical Immunology, 1985, 5, 287-97; Mandrup-Poulsen, T., Diabetes, 2001, 50, 558-563].

IL-8 is a chemotactic factor produced by various cell types including endothelial cells, mononuclear cells, fibroblasts and keratinocytes. IL-1, TNF and LPS all induce the production of IL-8 by endothelial cells. In vitro IL-8 has been shown to have a number of functions including being a chemoattractant for neutrophils, T-lymphocytes and basophils. IL-8 has also been shown to increase the surface expression of Mac-1 (CD11b/CD18) on neutrophils without de novo protein synthesis which may contribute to increased adhesion of neutrophils to vascular endothelial cells. Many diseases are characterised by massive neutrophil infiltration. Histamine release from basophils (in both atopic and normal individuals) is induced by IL-8 as is lysozomal enzyme release and respiratory burst from neutrophils.

The central role of IL-1 and TNF together with other leukocyte derived cytokines as important and critical inflammatory mediators is well documented. The inhibition of these cytokines has been shown or would be expected to be of benefit in controlling, alleviating or reducing many of these disease states.

The central position that p38 MAPK occupies within the cascade of signalling molecules mediating extracellular to intracellular signalling and its influence over not only IL-1, TNF and IL-8 production but also the synthesis and/or action of other pro-inflammatory proteins (e.g. IL-6, GM-CSF, COX-2, collagenase and stromelysin) make it an attractive target for inhibition by small molecule inhibitors with the expectation that such inhibition would be a highly effective mechanism for regulating the excessive and destructive activation of the immune system. Such an expectation is supported by the potent and diverse anti-inflammatory activities described for p38 MAPK inhibitors [Adams, ibid; Badger, et al., J. Pharm. Exp. Ther., 1996, 279, 1453-61; Griswold et al., Pharmacol. Comm., 1996, 7, 323-29].

We have now found a group of compounds which are potent and selective inhibitors of p38 MAPK (p38α, β, δ and γ) and the isoforms and splice variants thereof, especially p38α, p38β and p38β2. The compounds are thus of use in medicine, for example in the prophylaxis and treatment of immune or inflammatory disorders as described herein.

Thus according to one aspect of the invention we provide a compound of formula (1): 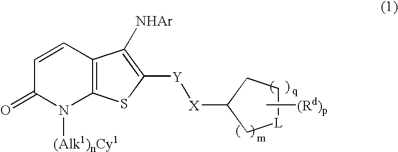
wherein:
X is a covalent bond or the group —N(R)—;
Y is a linking group —C(O)— or —S(O)₂—;
n is zero or the integer 1;
m is the integer 1, 2 or 3;
p is zero or the integer 1, 2, 3 or 4;
q is zero or the integer 1 or 2;
R is a hydrogen atom or a straight or branched C₁₋₆ alkyl group;
R^{d} is an —OH, -(Alk²)OH (where Alk² is a straight or branched C₁₋₄ alkylene chain), —OR¹ (where R¹ is a straight or branched C₁₋₆ alkyl group), -(Alk²)OR¹, —NR²R³ (where R² and R³ may be the same or different and is each independently a hydrogen atom or a straight or branched C₁₋₆ alkyl group), -(Alk²)NR²R³ or straight or branched C₁₋₆ alkyl group;
L is a linking atom or group —O—, —S—, —S(O)—, —S(O₂)—, —CH₂—, —CH(R^{d})—, —C(R^{d})₂— or —NR^{y}— where R^{y} is a hydrogen atom or a C₁₋₄ alkyl group;
Alk¹ is a straight or branched C₁₋₄ alkylene chain;
Cy¹ is an optionally substituted cycloaliphatic, polycycloaliphatic, heterocycloaliphatic, polyheterocycloaliphatic, aromatic or heteroaromatic group; and
Ar is an optionally substituted aromatic or heteroaromatic group;
and the salts, solvates, hydrates and N-oxides thereof.

The present invention also provides compounds wherein X is the group —N(R)—; p is the integer 1, 2, 3 or 4; and the remaining variables are as defined above.

It will be appreciated that compounds of formula (1) may have one or more chiral centres, and exist as enantiomers or diastereomers. The invention is to be understood to extend to all such enantiomers, diastereomers and mixtures thereof in any proportion, including racemates. Formula (1) and the formulae hereinafter are intended to represent all individual isomers and mixtures thereof, unless stated or shown otherwise. In addition, compounds of formula (1) may exist as tautomers, for example keto (CH₂C═O)-enol (CH═CHOH) tautomers. Formula (1) and the formulae hereinafter are intended to represent all individual tautomers and mixtures thereof, unless stated otherwise.

The following general terms as used herein in relation to compounds of the invention and intermediates thereto have the stated meaning below unless specifically defined otherwise.

Thus as used herein the term “alkyl” whether present as a group or part of a group includes straight or branched C₁₋₆alkyl groups, for example C₁₋₄alkyl groups such as methyl, ethyl, n-propyl, isopropyl, n-butyl, sec-butyl, isobutyl or tert-butyl groups. Similarly, the terms “alkenyl” or “alkynyl” are intended to mean straight or branched C₂₋₆alkenyl or C₂₋₆alkynyl groups such as C₂₋₄ alkenyl or C₂₋₄alkynyl groups. The optional substituents which may be present on these groups include one, two, three or more substituents where each substituent may be the same or different and is selected from halogen atoms, e.g. fluorine, chlorine, bromine or iodine atoms, or —OH, —CO₂H, —CO₂R⁴ [where R⁴ is an optionally substituted straight or branched C₁₋₆alkyl group, and is in particular a straight or branched C₁₋₄alkyl group], e.g. —CO₂CH₃ or —CO₂C(CH₃)₃, —CONHR⁴, e.g. —CONHCH₃, —CON(R⁴)₂, e.g. —CON(CH₃)₂, —COR⁴, e.g. —COCH₃, C₁₋₆alkoxy, e.g. methoxy or ethoxy, haloC₁₋₆alkoxy, e.g. trifluoromethoxy or difluoromethoxy, thiol (—SH), —S(O)R⁴, e.g. —S(O)CH₃, —S(O)₂R⁴, e.g. —S(O)₂CH₃, C₁₋₆alkylthio e.g. methylthio or ethylthio, amino, —NHR⁴, e.g. —NHCH₃, or —N(R⁴)₂, e.g. —N(CH₃)₂, groups. Where two R⁴ groups are present in any of the above substituents these may be the same or different.

In addition when two R⁴ alkyl groups are present in any of the optional substituents just described these groups may be joined, together with the N atom to which they are attached, to form a heterocyclic ring. Such heterocyclic rings may be optionally interrupted by a further heteroatom or heteroatom-containing group selected from —O—, —S—, —N(R⁴)—, —C(O)— or —C(S)— groups. Particular examples of such heterocyclic rings include piperidinyl, pyrazolidinyl, morpholinyl, thiomorpholinyl, pyrrolidinyl, imidazolidinyl and piperazinyl rings.

The term “halogen” is intended to include fluorine, chlorine, bromine or iodine atoms.

The term “haloalkyl” is intended to include those alkyl groups just mentioned substituted by one, two or three of the halogen atoms just described. Particular examples of such groups include —CF₃, —CCl₃, —CHF₂, —CHCl₂, —CH₂F and —CH₂Cl groups.

The term “alkoxy” as used herein is intended to include straight or branched C₁₋₆alkoxy, e.g. C₁₋₄alkoxy such as methoxy, ethoxy, n-propoxy, isopropoxy, n-butoxy, sec-butoxy, isobutoxy and tert-butoxy. “Haloalkoxy” as used herein includes any of these alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₆alkylthio, e.g. C₁₋₄alkylthio such as methylthio or ethylthio.

As used herein the term “alkylamino” or “dialkylamino” is intended to include the groups —NHR^{1a} and —N(R^{1a})(R^{1b}) where R^{1a} and R^{1b} is each independently an optionally substituted straight or branched alkyl group or both together with the N atom to which they are attached form an optionally substituted heterocycloalkyl group which may contain a further heteroatom or heteroatom-containing group such as an —O— or —S— atom or —N(R^{1a})— group.

Particular examples of such optionally substituted heterocycloalkyl groups include optionally substituted pyrrolidinyl, piperidinyl, morpholinyl, thiomorpholinyl and N′—C₁₋₆alkylpiperazinyl groups. The optional substituents which may be present on such heterocycloalkyl groups include those optional substituents as described above in relation to the term “alkyl”.

Particular examples of alkylene chains represented by Alk¹ and/or Alk² when each is present in compounds of the invention include —CH₂—, —CH₂CH₂—, —CH(CH₃)CH₂—, —(CH₂)₂CH₂—, —C(CH₃)₂—, —(CH₂)₃CH₂—, —CH₂CH(CH₃)CH₂—, —C(CH₃)₂CH₂— or —CH(CH₃)CH₂CH₂— chains.

Optionally substituted cycloaliphatic groups represented by the group Cy¹ in compounds of the invention include optionally substituted C₃₋₁₀cycloaliphatic groups. Particular examples include optionally substituted C₃₋₁₀cycloalkyl, e.g. C₃₋₇cycloalkyl, or C₃₋₁₀cycloalkenyl, e.g. C₃₋₇cycloalkenyl, groups.

Particular examples of cycloaliphatic groups represented by the group Cy¹ include optionally substituted cyclopropyl, cyclobutyl, cyclopentyl, cyclohexyl, cycloheptyl, 2-cyclobuten-1-yl, 2-cyclopenten-1-yl and 3-cyclopenten-1-yl groups.

The optional substituents which may be present on the cycloaliphatic, groups represented by the group Cy¹ include one, two, three or more substituents selected from halogen atoms, or C₁₋₆alkyl, e.g. methyl or ethyl, haloC₁₋₆alkyl, e.g. halomethyl or haloethyl such as difluoromethyl or trifluoromethyl, optionally substituted by hydroxyl, e.g. —C(OH)(CF₃)₂, C₁₋₆alkoxy, e.g. methoxy or ethoxy, haloC₁₋₆alkoxy, e.g. halomethoxy or haloethoxy such as difluoromethoxy or trifluoromethoxy, thiol, C₁₋₆alkylthiol, e.g. methylthiol or ethylthiol, carbonyl (═O), thiocarbonyl (═S), imino (═NR^{4a}) [where R^{4a} is an —OH group or a C₁₋₆alkyl group], or -(Alk³)ᵥR⁵ groups in which Alk³ is a straight or branched C₁₋₃alkylene chain, v is zero or the integer 1 and R⁵ is a C₃₋₈ cycloalkyl, —OH, —SH, —N(R⁶)(R⁷) [in which R⁶ and R⁷ is each independently selected from a hydrogen atom or an optionally substituted alkyl or C₃₋₈ cycloalkyl group], —OR⁶, —SR⁶, —CN, —NO₂, —CO₂R⁶, —SOR⁶, —SO₂R⁶, —SO₃R⁶, —OCO₂R⁶, —C(O)R⁶, —OC(O)R⁶, —C(S)R⁶, —C(O)N(R⁶)(R⁷), —OC(O)N(R⁶)(R⁷), —N(R⁶)C(O)R⁷, —C(S)N(R⁶)(R⁷), —N(R⁶)C(S)R⁷, —SO₂N(R⁶)(R⁷), —N(R⁶)SO₂R⁷, —N(R⁶)C(O)N(R⁷)(R⁸) [where R⁸ is as defined for R⁶], —N(R⁶)C(S)N(R⁷)(R⁸), —N(R⁶)SO₂N(R⁷)(R⁸) or an optionally substituted aromatic or heteroaromatic group.

Particular examples of Alk³ chains include —CH₂—, —CH₂CH₂—, —CH₂CH₂CH₂— and —CH(CH₃)CH₂— chains.

When R⁵, R⁶, R⁷ and/or R⁸ is present as a C₃₋₈cycloalkyl group it may be for example a cyclopropyl, cyclobutyl, cyclopentyl or cyclohexyl group. Optional substituents which may be present on such groups include for example one, two or three substituents which may be the same or different selected from halogen atoms, for example fluorine, chlorine, bromine or iodine atoms, or hydroxy or C₁₋₆alkoxy, e.g. methoxy, ethoxy or isopropoxy, groups.

When the groups R⁶ and R⁷ or R⁷ and R⁸ are both alkyl groups these groups may be joined, together with the N atom to which they are attached, to form a heterocyclic ring. Such heterocyclic rings may be optionally interrupted by a further heteroatom or heteroatom-containing group selected from —O—, —S—, —N(R⁷)—, —C(O)— or —C(S)— groups. Particular examples of such heterocyclic rings include piperidinyl, pyrazolidinyl, morpholinyl, thiomorpholinyl, pyrrolidinyl, imidazolidinyl and piperazinyl rings.

When R⁵ is an optionally substituted aromatic or heteroaromatic group it may be any such group as described hereinafter in relation to Cy¹.

In general, optionally substituted aromatic groups represented by the group Cy¹ include for example monocyclic or bicyclic fused ring C₆₋₁₂aromatic groups, such as phenyl, 1- or 2-naphthyl, 1- or 2-tetrahydronaphthyl, indanyl or indenyl groups, especially phenyl.

Heteroaromatic groups represented by the group Cy¹ include for example C₁₋₉ heteroaromatic groups containing for example one, two, three or four heteroatoms selected from oxygen, sulphur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulphur or nitrogen atoms. Bicyclic heteroaromatic groups include for example eight- to thirteen-membered fused ring heteroaromatic groups containing one, two or more heteroatoms selected from oxygen, sulphur or nitrogen atoms.

Particular examples of heteroaromatic groups of these types include pyrrolyl, furyl, thienyl, imidazolyl, N—C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,5-oxadiazolyl, 1,3,4-oxadiazolyl, 1,3,4-thiadiazolyl, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, benzothienyl, [2,3-dihydro]benzothienyl, benzotriazolyl, indolyl, indolinyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, quinoxalinyl, naphthyridinyl, imidazo[1,5-a]pyridinyl, imidazo[1,5-a]pyrazinyl, imidazo[1,5-c]pyrimidinyl, pyrido[3,4-b]pyridyl, pyrido[3,2-b]pyridyl, pyrido[4,3-b]pyridyl, quinolinyl, isoquinolinyl, phthalazinyl, tetrazolyl, 5,6,7,8-tetrahydroquinolinyl, 5,6,7,8-tetrahydroisoquinolinyl, imidyl, e.g. succinimidyl, phthalimidyl or naphthalimidyl such as 1,8-naphthalimidyl, pyrazolo[4,3-d]pyrimidinyl, furo[3,2-dopyrimidinyl, thieno[3,2-d]pyrimidinyl, pyrrolo[3,2-d]pyrimidinyl, pyrazolo[3,2-b]pyridinyl, furo[3,2-b]pyridinyl, thieno[3,2-b]pyridinyl, pyrrolo[3,2-b]pyridinyl, thiazolo[3,2-a]pyridinyl, pyrido[1,2-a]pyrimidinyl, tetrahydroimidazo[1,2-a]pyrimidinyl and dihydroimidazo[1,2-a]pyrimidinyl groups.

Optional substituents which may be present on aromatic or heteroaromatic groups represented by the group Cy¹ include one, two, three or more substituents, each selected from an atom or group R¹⁰ in which R¹⁰ is R^{10a} or -L⁶Alk⁵(R^{10a})ᵣ, where R^{10a} is a halogen atom, or an amino (—NH₂), substituted amino, nitro, cyano, hydroxyl (—OH), substituted hydroxyl, formyl, carboxyl (—CO₂H), esterified carboxyl, thiol (—SH), substituted thiol, —COR¹¹ [where R¹¹ is an -L⁶Alk³(R^{10a})ᵣ, aryl or heteroaryl group], —CSR¹¹, —SO₃H, —SOR¹¹, —SO₂R¹¹, —SO₃R¹¹, —SO₂NH₂, —SO₂NHR¹¹, —SO₂N(R¹¹)₂, —CONH₂, —CSNH₂, —CONHR¹¹, —CSNHR¹¹, —CON(R¹¹)₂, —CSN(R¹¹)₂, —N(R¹²)SO₂R¹¹ [where R¹² is a hydrogen atom or a straight or branched alkyl group], —N(SO₂R¹¹)₂, —N(R¹²)SO₂NH₂, —N(R¹²)SO₂NH R¹¹, —N(R¹²)SO₂N(R¹¹)₂, —N(R¹²)COR¹¹, —N(R¹²)CONH₂, —N(R¹²)CONHR¹¹, —N(R¹²)CON(R¹¹)₂, —N(R¹²)CSNH₂, —N(R¹²)CSNHR¹¹, —N(R¹²)CSN(R¹¹)₂, —N(R¹²)CSR₁₁—N(R¹²)C(O)OR¹¹, —C═NR¹²(NR¹²), —SO₂NHet¹ [where —NHet¹ is an optionally substituted C₃₋₇ cyclicamino group optionally containing one or more other —O— or —S— atoms or —N(R¹²)—, —C(O)— or —C(S)— groups], —CONHet¹, —CSNHet¹, —N(R¹²)SO₂NHet¹, —N(R¹²)CONHet¹, —N(R¹²)CSNHet¹, —SO₂N(R¹²)Het [where -Het is an optionally substituted monocyclic C₃₋₇carbocyclic group optionally containing one or more other —O— or —S— atoms or —N(R¹²)—, —C(O)—, —S(O)— or —S(O)₂— groups], -Het, —CON(R¹²)Het, —CSN(R¹²)Het, —N(R¹²)CON(R¹²)Het, —N(R¹²)CSN(R¹²)Het, —N(R¹²)SO₂N(R¹²)Het, aryl or heteroaryl groups; L⁶ is a covalent bond or a linker atom or group; Alk⁵ is an optionally substituted straight or branched C₁₋₆alkylene, C₂₋₆alkenylene or C₂₋₆alkynylene chain, optionally interrupted by one, two or three —O— or —S— atoms or —S(O)ₖ— [where k is an integer 1 or 2] or —N(R¹²)—, e.g. —N(CH₃)—, groups; and r is zero or the integer 1, 2, or 3. It will be appreciated that when two R¹¹ or R¹² groups are present in one of the above substituents the R¹¹ and R¹² groups may be the same or different.

When L⁶ in the group -L⁶Alk⁵(R^{10a})ᵣ is a linker atom or group it may be for example any divalent linking atom or group. Particular examples include —O— or —S— atoms or —C(O)—, —C(O)O—, —OC(O)—, —C(S)—, —S(O)—, —S(O)₂—, —N(R³)— [where R³ is a hydrogen atom or a straight or branched alkyl group], —N(R³)O—, —N(R³)N—, —CON(R³)—, —OC(O)N(R³)—, —CSN(R³)—, —N(R³)CO—, —N(R³)C(O)O—, —N(R³)CS—, —S(O)₂N(R³)—, —N(R³)S(O)₂—, —N(R³)CON(R³)—, —N(R³)CSN(R³)— or —N(R³)SO₂N(R³)— groups. Where L⁶ contains two R³ groups these may be the same or different.

When in the group -L⁶Alk⁵(R^{10a})ᵣ r is an integer 1, 2 or 3, it is to be understood that the substituent or substituents R^{10a} may be present on any suitable carbon atom in -Alk⁵. Where more than one R^{10a} substituent is present these may be the same or different and may be present on the same or different atom in -Alk⁵. Clearly, when r is zero and no substituent R^{10a} is present the alkylene, alkenylene or alkynylene chain represented by Alk⁵ becomes an alkyl, alkenyl or alkynyl group.

When R^{10a} is a substituted amino group it may be for example a group —NHR¹¹ [where R¹¹ is as defined above) or a group —N(R¹¹)₂ wherein each R¹¹ group is the same or different.

When R^{10a} is a halogen atom it may be for example a fluorine, chlorine, bromine, or iodine atom.

When R^{10a} is a substituted hydroxyl or substituted thiol group it may be for example a group —OR¹¹ or —SR¹² respectively.

Esterified carboxyl groups represented by the group R^{10a} include groups of formula —CO₂Alk⁶ wherein Alk⁶ is a straight or branched, optionally substituted C₁-alkyl group such as a methyl, ethyl, n-propyl, isopropyl, n-butyl, isobutyl, sec-butyl or tert-butyl group; a C₆₋₁₂arylC₁₋₈alkyl group such as an optionally substituted benzyl, phenylethyl, phenylpropyl, 1-naphthylmethyl or 2-naphthylmethyl group; a C₆₋₂aryl group such as an optionally substituted phenyl, 1-naphthyl or 2-naphthyl group; a C₆₋₁₂aryloxyC₁₋₈alkyl group such as an optionally substituted phenyloxymethyl, phenyloxyethyl, 1-naphthyloxymethyl, or 2-naphthyloxymethyl group, an optionally substituted C₁-alkanoyloxyC₁₋₈alkyl group, such as a pivaloyloxymethyl, propionyloxyethyl or propionyloxypropyl group; or a C₆₋₁₂aroyloxyC₁₋₈alkyl group such as an optionally substituted benzoyloxyethyl or benzoyloxypropyl group. Optional substituents, present on the Alk⁶ group, include R^{10a} atoms and groups as described above.

When Alk⁵ is present in or as a substituent it may be for example a —CH₂—, —CH(CH₃)—, —C(CH₃)₂, —CH₂CH₂—, —CH₂CH₂CH₂—, —CH(CH₃)CH₂—, —CH₂CH₂CH₂CH₂—, —CH₂CH(CH₃)CH₂—, —CH(CH₃)CH₂CH₂—, —C(CH₃)₂CH₂—, —CH═CH—, —CH═CHCH₂—, —CH₂CH═CH—, —CH═CHCH₂CH₂—, —CH₂CH═CHCH₂—, —CH₂CH₂CH═CH—, —C═C—, —C≡CCH₂—, —CH₂C≡C—, —C≡CCH₂CH₂—, —CH₂C≡CCH₂— or —CH₂CH₂C≡C— chain, optionally interrupted by one, two, or three —O— or —S— atoms or —S(O)—, —S(O)₂— or —N(R¹²)—, e.g. —N(CH₃)—, groups. The aliphatic chains represented by Alk⁵ may be optionally substituted by one, two or three halogen atoms in addition to any R^{10a} groups that may be present.

Aryl or heteroaryl groups represented by the groups R^{10a} or R¹¹ include mono- or bicyclic optionally substituted C₆₋₁₂ aromatic or C₁₋₉ heteroaromatic groups as described above for the group Cy¹. The aromatic and heteroaromatic groups may be attached to the group Cy¹ in compounds of formula (1) by any carbon atom or heteroatom, e.g. nitrogen atom, as appropriate.

It will be appreciated that when —NHet¹ or -Het forms part of a substituent R¹⁰ the heteroatoms or heteroatom-containing groups that may be present within the ring —NHet¹ or -Het take the place of carbon atoms within the parent carbocyclic ring.

Thus when —NHet¹ or -Het forms part of a substituent R¹⁰ each may be for example an optionally substituted pyrrolidinyl, imidazolidinyl, pyrazolidinyl, piperazinyl, morpholinyl, thiomorpholinyl, piperidinyl or thiazolidinyl group. Additionally Het may represent, for example, an optionally substituted cyclopentyl or cyclohexyl group. Optional substituents which may be present on —NHet¹ include those substituents described above when Cy¹ is a heterocycloaliphatic group.

Particularly useful atoms or groups represented by R¹⁰ include fluorine, chlorine, bromine or iodine atoms, or C₁₋₆alkyl, e.g. methyl, ethyl, n-propyl, isopropyl, n-butyl or tert-butyl, optionally substituted phenyl, pyridyl, pyrimidinyl, pyrrolyl, furyl, thiazolyl, or thienyl, C₁₋₆hydroxyalkyl, e.g. hydroxymethyl or hydroxyethyl, carboxyC₁₋₆alkyl, e.g. carboxyethyl, C₁₋₆alkylthio, e.g. methylthio or ethylthio, carboxyC₁₋₆alkylthio, e.g. carboxymethylthio, 2-carboxyethylthio or 3-carboxy-propylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₃₋₇cycloalkyl, e.g. cyclobutyl, cyclopentyl, C₁₋₇ cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, —CH(CH₃)NH₂ or —C(CH₃)₂NH₂, haloC₁₋₆alkylamino, e.g. fluoroC₁₋₆alkylamino, —CH(CF₃)NH₂ or —C(CF₃)₂NH₂, amino (—NH₂), aminoC₁₋₆alkyl, e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkyl-aminoC₁₋₆alkoxy, e.g. dimethylaminoethoxy, diethylaminoethoxy, diisopropylaminoethoxy or dimethylaminopropoxy, imido, such as phthalimido or naphthalimido, e.g. 1,8-naphthalimido, nitro, cyano, hydroxyl (—OH), formyl [HC(O)—], carboxyl (—CO₂H), —CO₂Alk⁶ [where Alk⁶ is as defined above], C₁₋₆ alkanoyl, e.g. acetyl, optionally substituted benzoyl, thiol (—SH), thioC₁₋₆alkyl, e.g. thiomethyl or thioethyl, sulphonyl (—SO₃H), C₁₋₆alkylsulphonyl, e.g. methylsulphonyl, aminosulphonyl (—SO₂NH₂), C₁₋₆alkylaminosulphonyl, e.g. methylaminosulphonyl or ethylaminosulphonyl, C₁₋₆dialkylaminosulphonyl, e.g. dimethylaminosulphonyl or diethylaminosulphonyl, phenylaminosulphonyl, carboxamido (—CONH₂), C₁₋₆alkylaminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylamino-carbonyl, C₁₋₆dialkylaminoC₁₋₆alkylaminocarbonyl, e.g. diethylaminoethyl-aminocarbonyl, aminocarbonylamino, C₁₋₆alkylaminocarbonylamino, e.g. methylaminocarbonylamino or ethylaminocarbonylamino, C₁₋₆dialkylamino-carbonylamino, e.g. dimethylaminocarbonylamino or diethylamino-carbonylamino, C₁₋₆alkylaminocabonylC₁-alkylamino, e.g. methylamino-carbonylmethylamino, aminothiocarbonylamino, C₁₋₆alkylaminothiocarbonyl-amino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₄alkylamino, e.g. ethylaminothiocarbonylmethylamino, —CONHC(═NH)NH₂, C₁-alkylsulphonylamino, e.g. methylsulphonylamino or ethylsulphonylamino, C₁₋₆dialkylsulphonylamino, e.g. dimethylsulphonylamino or diethylsulphonylamino, optionally substituted phenylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino, e.g. aminoacetylamino, C₁-dialkylaminoC₁₋₆alkanoylamino, e.g. dimethylaminoacetylamino, C₁-alkanoylaminoC₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or tert-butoxycarbonylamino, or optionally substituted benzyloxy, pyridylmethoxy, thiazolylmethoxy, benzyloxycarbonylamino, benzyloxycarbonylaminoC₁₋₆alkyl, e.g. benzyloxycarbonylaminoethyl, benzothio, pyridylmethylthio or thiazolylmethylthio groups.

A further particularly useful group of substituents represented by R¹⁰ when present on aromatic or heteroaromatic groups includes substituents of formula -L⁶Alk⁵R^{10a} where L⁶ is preferably a covalent bond or an —O— or —S— atom or —N(R³), —C(O)—, —C(O)O—, —O—C(O)—, —N(R³)CO—, —CON(R³) or —N(R³)S(O)₂— group, Alk⁵ is an optionally substituted C₁₋₆alkyl group optionally interrupted by one or two —O— or —S— atoms or —N(R¹²)—, —C(O)—, —C(S)—, —CON(R¹²)— or —N(R¹²)CO— groups, and R^{10a} is an optionally substituted Het group as herein defined or an optionally substituted heteroaromatic group as hereinbefore described in relation to Cy¹.

Where desired, two R¹⁰ substituents may be linked together to form a cyclic group such as a cyclic ether, e.g. a C₁₋₆alkylenedioxy group such as methylenedioxy or ethylenedioxy.

It will be appreciated that where two or more R¹⁰ substituents are present, these need not necessarily be the same atoms and/or groups. In general, the substituent(s) may be present at any available ring position on the aromatic or heteroaromatic group represented by the group Cy¹.

The substituted aromatic or heteroaromatic group represented by Ar in compounds of the invention may be any aromatic or heteroaromatic group as hereinbefore described for Cy¹. Optional substituents which may be present include those R¹⁰ atoms and groups as generally or particularly described in relation to Cy¹ aromatic and heteroaromatic groups.

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulfonates, e.g. methanesulfonates, ethanesulfonates, or isothionates, arylsulfonates, e.g. p-toluenesulfonates, besylates or napsylates, phosphates, sulphates, hydrogensulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

In one embodiment, X is the group —N(R)—. In another embodiment, X is a covalent bond.

In a preferred embodiment, Y is a —C(O)— group. In an alternative embodiment, Y is a —S(O)₂— group.

In one class of compounds of formula (1) n is the integer 1. When in compounds of formula (1) n is the integer 1, Alk¹ is preferably a —CH₂CH₂-chain or more especially is —CH₂—.

In one class of compounds of formula (1) n is zero.

Particularly preferred Cy¹ optionally substituted cycloaliphatic groups include optionally substituted C₃₋₇cycloalkyl groups, especially cyclopropyl, cyclobutyl, cyclopentyl or cyclohexyl groups. Cy¹ is in particular a cyclopropyl group.

Each of these preferred Cy¹ cycloalkyl groups may be unsubstituted. When substituents are present these may in particular include halogen atoms, especially fluorine, chlorine or bromine atoms, or C₁₋₆alkyl groups, especially C₁₋₃alkyl groups, most especially a methyl group, or haloC₁₋₆alkyl groups, especially fluoroC₁₋₆alkyl groups, most especially a —CF₃ group, or C₁₋₆alkoxy groups, especially a methoxy, ethoxy, propoxy or isopropoxy group, or haloC₁₋₆alkoxy groups, especially fluoroC₁₋₆alkoxy groups, most especially a —OCF₃ group, or a cyano (—CN), esterified carboxyl, especially —CO₂CH₃ or —CO₂C(CH₃)₃, nitro (—NO₂), amino (—NH₂), substituted amino, especially —NHCH₃ or —N(CH₃)₂, —C(O)R⁶, especially —C(O)CH₃, or —N(R⁶)C(O)R⁷, especially —NHCOCH₃, group.

Particularly preferred Cy¹ aromatic groups include optionally substituted phenyl groups. Particularly preferred heteroaromatic groups include optionally substituted monocyclic heteroaromatic groups, especially optionally substituted five- or six-membered heteroaromatic groups containing one, two, three or four heteroatoms selected from oxygen, sulphur or nitrogen atoms. Particularly preferred optionally substituted monocyclic heteroaromatic groups include optionally substituted furyl, thienyl, pyrrolyl, oxazolyl, thiazolyl, pyridyl, pyrimidinyl or triazinyl groups. In a further preference, the heteroaromatic group may be an eight- to thirteen-membered bicyclic fused ring containing one or two oxygen, sulphur or nitrogen atoms. Particularly useful groups of this type include optionally substituted indolyl groups.

Particularly preferred optional substituents which may be present on Cy, aromatic or heteroaromatic groups include one, two or three atoms or groups —R^{10a} or -L⁶Alk⁵(R^{10a})ᵣ as hereinbefore defined. Particularly useful optional substituents include halogen atoms, especially fluorine, chlorine or bromine atoms, or C₁₋₆alkyl groups, especially C₁₋₃alkyl groups, most especially a methyl group, or haloC₁₋₆alkyl groups, especially fluoroC₁₋₆alkyl groups, most especially a —CF₃ group, or C₁₋₆alkoxy groups, especially a methoxy, ethoxy, propoxy or isopropoxy group, or haloC₁₋₆alkoxy groups, especially fluoroC₁₋₆alkoxy groups, most especially a —OCF₃ group, or a cyano (—CN), carboxyl (—CO₂H), esterified carboxyl (—CO₂Alk⁶), especially —CO₂CH₃, —CO₂CH₂CH₃, or —CO₂C(CH₃)₃, nitro (—NO₂), amino (—NH₂), substituted amino, especially —NHCH₃ or —N(CH₃)₂, —COR¹¹, especially —COCH₃, or —N(R¹²)COR¹¹, especially —NHCOCH₃, group.

Further preferred optional substituents which may be present on Cy¹ aromatic or heteroaromatic groups include groups of formula -L⁶Alk⁵(R^{10a})ᵣ in which r is the integer 1 or 2, L⁶ is a covalent bond or an —O— or —S— atom or a —N(R³)—, especially —NH— or —N(CH₃)—, —C(O)—, —C(S)—, —C(O)O—, —OC(O)—, —N(R³)CO—, especially —NHCO—, or —CON(R³)—, especially —CONH—, group, Alk⁵ is a C₁₋₆alkylene chain, especially a —CH₂—, —CH₂CH₂—, —CH₂CH₂CH₂— or —CH₂CH₂CH₂CH₂— chain, and R^{10a} is a hydroxyl or substituted hydroxyl group, especially a —OCH₃, —OCH₂CH₃ or —OCH(CH₃)₂ group, or a —NH₂ or substituted amino group, especially a —N(CH₃)₂ or —N(CH₂CH₃)₂ group, or a -Het group, especially an optionally substituted monocyclic C₅₋₇carbocyclic group containing one, two or three —O—, —S—, —N(R¹²)—, especially —NH— or —N(CH₃)—, or —C(O)— groups within the ring structure as previously described, most especially an optionally substituted pyrrolidinyl, imidazolidinyl, piperidinyl, e.g. N-methylpiperidinyl, morpholinyl, thiomorpholinyl or piperazinyl group, or R^{10a} is an optionally substituted heteroaromatic group, especially a five- or six-membered monocyclic heteroaromatic group containing one, two, three or four heteroatoms selected from oxygen, sulphur or nitrogen atoms, such as optionally substituted pyrrolyl, furyl, thienyl, imidazolyl, triazolyl, pyridyl, pyrimidinyl, triazinyl, pyridazinyl, or pyrazinyl group. Particularly preferred optional substituents on the -Het groups just described include hydroxyl (—OH) and carboxyl (—CO₂H) groups or those preferred optional substituents just described in relation to the group Cy¹, especially when Cy¹ is a cycloalkyl group.

In one particularly preferred group of compounds of formula (1) Cy¹ is an optionally substituted phenyl group, especially a phenyl group optionally substituted by one, two or three substituents where at least one, and preferably two, substituents are located ortho to the bond joining Cy¹ to the remainder of the compound of formula (1). Particularly preferred ortho substituents include halogen atoms, especially fluorine or chlorine atoms, or C₁₋₃alkyl groups, especially methyl, C₁₋₃alkoxy groups, especially methoxy, haloC₁₋₃alkyl groups, especially —CF₃, haloC₁₋₃alkoxy groups, especially —OCF₃, or cyano (—CN), groups. In this class of compounds a second or third optional substituent when present in a position other than the ortho positions of the ring Cy¹ may be preferably an atom or group —R^{10a} or -L⁶Alk⁵(R^{10a})ᵣ as herein generally and particularly described. In another preference, the Cy¹ phenyl group may have a substituent para to the bond joining Cy¹ to the remainder of the compound of formula (1). Particular para substituents include those particularly preferred ortho substituents just described. Where desired, the para substituent may be present with other ortho or meta substituents as just mentioned.

A particular Cy¹ group is phenyl.

Particularly preferred Ar aromatic groups in compounds of formula (1) include optionally substituted phenyl groups. Particularly preferred heteroaromatic groups include optionally substituted monocyclic heteroaromatic groups, especially optionally substituted five- or six-membered heteroaromatic groups containing one, two, three or four heteroatoms selected from oxygen, sulphur or nitrogen atoms. Particularly preferred optionally substituted monocyclic heteroaromatic groups include optionally substituted furyl, thienyl, pyrrolyl, oxazolyl, thiazolyl, pyridyl, pyrimidinyl and triazinyl groups.

Particularly preferred optional substituents which may be present on Ar aromatic or heteroaromatic groups include atoms or groups —R^{10a} or -L⁶Alk⁵(R^{10a})ᵣ as hereinbefore defined. Particularly useful optional substituents include halogen atoms, especially fluorine, chlorine or bromine atoms, or C₁₋₆alkyl groups, especially C₁₋₃alkyl groups, most especially a methyl group, or haloC₁₋₆alkyl groups, especially fluoroC₁₋₆alkyl groups, most especially a —CF₃ group, or C₁₋₆alkoxy groups, especially a methoxy, ethoxy, propoxy or isopropoxy group, or haloC₁₋₆alkoxy groups, especially fluoroC₁₋₆alkoxy groups, most especially a —OCF₃ group, or a cyano (—CN), esterified carboxyl, especially —CO₂CH₃ or —CO₂C(CH₃)₃, nitro (—NO₂), amino (—NH₂), substituted amino, especially —NHCH₃ or —N(CH₃)₂, —COR¹¹, especially —COCH₃, or —N(R¹²)COR¹¹, especially —NHCOCH₃, group.

Particularly useful Ar groups in compounds of formula (1) include phenyl and mono- or disubstituted phenyl groups in which each substituent is in particular a —R^{10a} or -L⁶Alk⁵(R^{10a})ᵣ atom or group as just defined and is especially a halogen atom or a C₁₋₃alkyl, C₁₋₃alkoxy or —CN group.

Examples of particular substituents on Ar include halogen and C₁₋₆ alkyl, especially fluoro or methyl.

Examples of specific substituents on Ar include halogen, especially fluoro.

Particular Ar groups include phenyl, difluorophenyl (especially 2,4-difluorophenyl), (fluoro)(methyl)phenyl (especially 4-fluoro-3-methylphenyl) and methylpyridinyl (especially 6-methylpyridin-2-yl).

Specific Ar groups include phenyl and difluorophenyl (especially 2,4-difluorophenyl).

Particular examples of Alk² when present in compounds of the invention include —CH₂—, —CH₂CH₂—, —C(CH₃)₂— and —CH(CH₃)CH₂—.

Suitably, R¹ represents hydrogen or methyl. In one embodiment, R¹ is hydrogen. In another embodiment, R¹ is methyl.

Suitably, R² represents hydrogen or methyl. In one embodiment, R² is hydrogen. In another embodiment, R² is methyl.

Suitably, R³ represents hydrogen or methyl. In one embodiment, R³ is hydrogen. In another embodiment, R³ is methyl.

The group R in compounds of formula (1) is preferably a hydrogen atom.

Suitably, R^{y} represents hydrogen or methyl. In one embodiment, R^{y} is hydrogen. In another embodiment, R^{y} is methyl.

L in compounds of the invention is in particular a —CH₂—, —CH(R^{d})_, —NH— or —N(CH₃)— group. In one embodiment, L is —CH₂—. In another embodiment, L is —NH—. In a further embodiment, L is —N(CH₃)—.

In compounds of the invention, m and q may be selected to vary the ring size from a ring having any number of total ring members from 4 up to 8 inclusive. Particularly advantageous rings are those wherein m and q are selected to provide rings having a total of 4, 5 or 6 members.

In one embodiment, m is the integer 1. In another embodiment, m is the integer 2.

In one embodiment, q is zero. In another embodiment, q is the integer 1.

In one embodiment, p is zero. In another embodiment, p is the integer 1.

Each substituent R^{d} may be present on any ring carbon atom. In one particular class of compounds of the invention one or two R^{d} substituents are present.

Particular R^{d} substituents include —OH, —CH₂OH, —CH(CH₃)OH and —C(CH₃)₂OH groups.

In a specific embodiment, R^{d} is —OH.

Particularly useful compounds of the invention include each of the compounds described in the Examples hereinafter, and the salts, solvates, hydrates and N-oxides thereof.

Compounds according to the invention are potent and selective inhibitors of p38 MAPKs, including all isoforms and splice variants thereof. More specifically the compounds of the invention are inhibitors of p38α, p38β and p38β2. The ability of the compounds to act in this way may be simply determined by employing tests such as those described in the Examples hereinafter.

The compounds of formula (1) are of use in modulating the activity of p38 MAPKs and in particular are of use in the prophylaxis and treatment of any p38 MAPK mediated diseases or disorders in a human, or other mammal. The invention extends to such a use and to the use of the compounds for the manufacture of a medicament for treating such diseases or disorders. Further the invention extends to the administration to a human of an effective amount of a p38 MAPK inhibitor for treating any such disease or disorder.

The invention also extends to the prophylaxis or treatment of any disease or disorder in which p38 MAPK plays a role including conditions caused by excessive or unregulated pro-inflammatory cytokine production including for example excessive or unregulated TNF, IL-1, IL-6 and IL-8 production in a human, or other mammal. The invention extends to such a use and to the use of the compounds for the manufacture of a medicament for treating such cytokine-mediated diseases or disorders. Further the invention extends to the administration to a human of an effective amount of a p38 MAPK inhibitor for treating any such disease or disorder.

Diseases or disorders in which p38 MAPK plays a role either directly or via pro-inflammatory cytokines including the cytokines TNF, IL-1, IL-6 and IL-8 include without limitation autoimmune diseases, inflammatory diseases, destructive bone disorders, proliferative disorders, neurodegenerative disorders, viral diseases, allergies, infectious diseases, heart attacks, angiogenic disorders, reperfusion/ischemia in stroke, vascular hyperplasia, organ hypoxia, cardiac hypertrophy, thrombin-induced platelet aggregation and conditions associated with prostaglandin endoperoxidase synthetase-2 (COX-2).

Autoimmune diseases which may be prevented or treated include but are not limited to rheumatoid arthritis, inflammatory bowel disease, ulcerative colitis, Crohn's disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs, host disease and psoriasis.

The invention further extends to the particular autoimmune disease rheumatoid arthritis.

Inflammatory diseases which may be prevented or treated include but are not limited to asthma, allergies, respiratory distress syndrome and acute or chronic pancreatitis.

Destructive bone disorders which may be prevented or treated include but are not limited to osteoporosis, osteoarthritis and multiple myeloma-related bone disorder.

Proliferative diseases which may be prevented or treated include but are not limited to acute or chronic myelogenous leukemia, Kaposi's sarcoma, metastatic melanoma and multiple myeloma.

Neurodegenerative diseases which may be prevented or treated include but are not limited to Parkinson's disease, Alzheimer's disease, cerebral ischemias and neurodegenerative disease caused by traumatic injury.

Viral diseases which may be prevented or treated include but are not limited to acute hepatitis infection (including hepatitis A, hepatitis B and hepatitis C), HIV infection and CMV retinitis.

Infectious diseases which may be prevented or treated include but are not limited to septic shock, sepsis and Shigellosis.

In addition, p38 MAPK inhibitors of this invention also exhibit inhibition of expression of inducible pro-inflammatory proteins such as prostaglandin endoperoxidase synthetase-2, otherwise known as cyclooxygenase-2 (COX-2), and are therefore of use in therapy. Pro-inflammatory mediators of the cyclooxygenase pathway derived from arachidonic acid are produced by inducible COX-2 enzyme. Regulation of COX-2 would regulate these pro-inflammatory mediators such as prostaglandins, which affect a wide variety of cells and are important and critical inflammatory mediators of a wide variety of disease states and conditions. In particular these inflammatory mediators have been implicated in pain, such as in the sensitization of pain receptors, or edema. Accordingly, additional p38 MAPK mediated conditions which may be prevented or treated include edema, analgesia, fever and pain such as neuromuscular pain, headache, dental pain, arthritis pain and pain caused by cancer.

As a result of their p38 MAPK inhibitory activity, compounds of the invention have utility in the prevention and treatment of diseases associated with cytokine production including but not limited to those diseases associated with TNF, IL-1, IL-6 and IL-8 production.

Thus, TNF mediated diseases or conditions include for example rheumatoid arthritis, rheumatoid spondylitis, osteoarthritis, gouty arthritis and other arthritic conditions, sepsis, septic shock syndrome, adult respiratory distress syndrome, cerebral malaria, chronic pulmonary inflammatory disease, silicosis, pulmonary sarcoidosis, bone resorption disease, reperfusion injury, graft vs. host reaction, allograft rejections, fever and myalgias due to infection, cachexia secondary to infection, AIDS, ARC or malignancy, keloid formation, scar tissue formation, Crohn's disease, ulcerative colitis, pyresis, viral infections such as HIV, CMV, influenza and herpes; and veterinary viral infections, such as lentivirus infections, including but not limited to equine infectious anaemia virus, caprine arthritis virus, visna virus or maedi virus; or retrovirus infections, including feline immunodeficiency virus, bovine immunodeficiency virus and canine immunodeficiency virus.

Compounds of the invention may also be used in the treatment of viral infections, where such viruses elicit TNF production in vivo or are sensitive to upregulation by TNF. Such viruses include those that produce TNF as a result of infection and those that are sensitive to inhibition, for instance as a result of decreased replication, directly or indirectly by the TNF inhibiting compounds of the invention. Such viruses include, but are not limited to, HIV-1, HIV-2 and HIV-3, Cytomegalovirus (CMV), Influenza, adenovirus and the Herpes group of viruses such as Herpes Zoster and Herpes Simplex.

IL-1 mediated diseases or conditions include for example rheumatoid arthritis, osteoarthritis, psoriatic arthritis, traumatic arthritis, rubella arthritis, inflammatory bowel disease, stroke, endotoxemia and/or toxic shock syndrome, inflammatory reaction induced by endotoxin, diabetes, pancreatic β-cell disease, Alzheimer's disease, tuberculosis, atherosclerosis, muscle degeneration and cachexia.

IL-8 mediated diseases and conditions include for example those characterized by massive neutrophil infiltration such as psoriasis, inflammatory bowel disease, asthma, cardiac, brain and renal reperfusion injury, adult respiratory distress syndrome, thrombosis and glomerulonephritis. The increased IL-8 production associated with each of these diseases is responsible for the chemotaxis of neutrophils into inflammatory sites. This is due to the unique property of IL-8 (in comparison to TNF, IL-1 and IL-6) of promoting neutrophil chemotaxis and activation. Therefore, inhibition of IL-8 production would lead to a direct reduction in neutrophil infiltration.

It is also known that both IL-6 and IL-8 are produced during rhinovirus (HRV) infections and contribute to the pathogenesis of the common cold and exacerbation of asthma associated with HRV infection [Turner et al., Clin. Infec. Dis., 1997, 26, 840; Grunberg et al., Am. J. Cit. Care Med., 1997, 155, 1362; Zhu et al., J. Clin. Invest., 1996, 97, 421]. It has also been demonstrated in vitro that infection of pulmonary epithelial cells (which represent the primary site of infection by HRV) with HRV results in production of IL-6 and IL-8 [Sabauste et al., J. Clin. Invest., 1995, 96, 549]. Therefore, p38 MAPK inhibitors of the invention may be used for the treatment or prophylaxis of the common cold or respiratory viral infection caused by human rhinovirus infection (HRV), other enteroviruses, coronavirus, influenza virus, parainfluenza virus, respiratory syncytial virus or adenovirus infection.

For the prophylaxis or treatment of a p38 MAPK or pro-inflammatory cytokine mediated disease the compounds according to the invention may be administered to a human or mammal as pharmaceutical compositions, and according to a further aspect of the invention we provide a pharmaceutical composition which comprises a compound of formula (1) together with one or more pharmaceutically acceptable carriers, excipients or diluents.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

The compounds of formula (1) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (1) may also be formulated as a depot preparation. Such long-acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichlorofluoromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds for use according to the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively the compounds for use according to the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds for use according to the present invention may be conveniently formulated as microionized suspensions in isotonic, pH adjusted sterile saline, either with or without a preservative such as a bactericidal or fungicidal agent, for example phenylmercuric nitrate, benzylalkonium chloride or chlorhexidine acetate.

Alternatively for ophthalmic administration compounds may be formulated in an ointment such as petrolatum.

For rectal administration the compounds for use according to the present invention may be conveniently formulated as suppositories. These can be prepared by mixing the active component with a suitable non-irritating excipient which is solid at room temperature but liquid at rectal temperature and so will melt in the rectum to release the active component. Such materials include for example cocoa butter, beeswax and polyethylene glycols.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg, e.g. around 0.01 mg/kg to 40 mg/kg body weight, for oral or buccal administration, from around 10 ng/kg to 50 mg/kg body weight for parenteral administration, and around 0.05 mg to around 1000 mg, e.g. around 0.5 mg to around 1000 mg, for nasal administration or administration by inhalation or insufflation.

The compounds of the invention may be prepared by a number of processes as generally described below and more specifically in the Examples hereinafter. In the following process description, the symbols Ar, Cy¹, Alk¹, n, R, R^{d}, p, m, q, Y and L when used in the formulae depicted are to be understood to represent those groups described above in relation to formula (1) unless otherwise indicated. In the reactions described below, it may be necessary to protect reactive functional groups, for example hydroxy, amino, thio or carboxy groups, where these are desired in the final product, to avoid their unwanted participation in the reactions. Conventional protecting groups may be used in accordance with standard practice [see, for example, Greene, T. W. in “Protective Groups in Organic Synthesis”, John Wiley and Sons, 1999]. In some instances, deprotection may be the final step in the synthesis of a compound of formula (1) and the processes according to the invention described hereinafter are to be understood to extend to such removal of protecting groups.

Thus, according to a further aspect of the invention a compound of formula (1) in which X is —N(R)— and Y is a —C(O)— group may be prepared from a carboxylic acid of formula (2) or ester of formula (5) according to amide bond forming reactions well known to those skilled in the art. Such reactions are set forth in references such as March's Advanced Organic Chemistry (John Wiley and Sons 1992), Larock's Comprehensive Organic Transformations (VCH Publishers Inc., 1992) and Comprehensive Organic Functional Group Transformations, ed. Katritzky et al., volumes 1-8, 1984, and Volumes 1-11, 1994 (Pergamon). Examples of such methods that may be employed to give compounds of formula (1a) are set out, but not limited to the reactions, in Scheme 1 and Scheme 2 below.

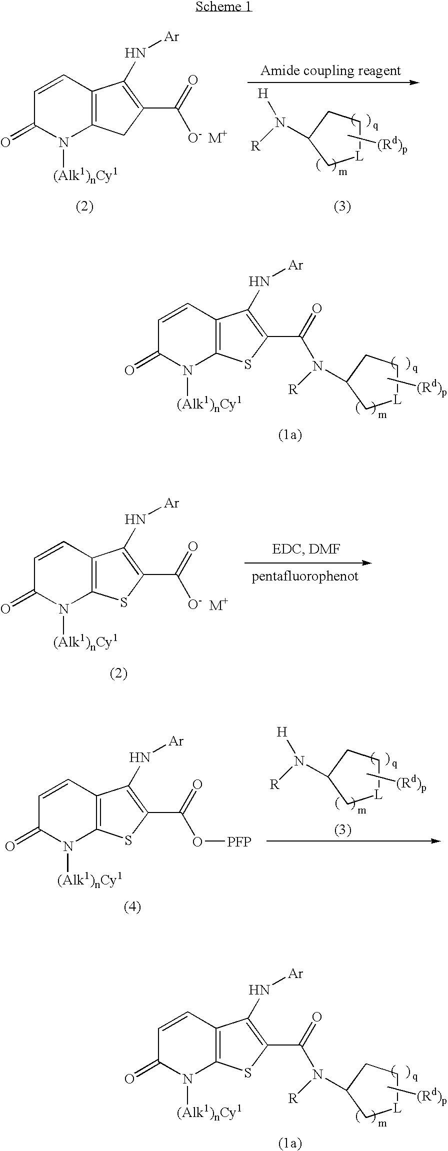

Thus, amides of formula (1a) may be formed by reaction of a carboxylate salt of formula (2) [where M⁺ is metal counterion such as a sodium or lithium ion or is alternatively an ammonium or trialkylammonium counterion] with an amine of formula (3) in the presence of a coupling reagent such as a carbodiimide, e.g. 1-(3-dimethylaminopropyl)-3-ethylcarbodiimide (EDC) or N,N′-dicyclohexylcarbodiimide, optionally in the presence of a base such as an amine, e.g. triethylamine or N-methylmorpholine. These reactions may be performed in a solvent such as an amide solvent, e.g. N,N-dimethylformamide (DMF), or an ether, e.g. a cyclic ether such as tetrahydrofuran or 1,4-dioxane, or a halogenated solvent such as dichloromethane, at around ambient temperature to 60° C. In another procedure a pentafluorophenyl ester of formula (4) may be prepared by reaction of a carboxylic acid of formula (2) with pentafluorophenol in the presence of a coupling reagent such as 1-(3-dimethylaminopropyl)-3-ethylcarbodiimide in a solvent such as an amide solvent, e.g. DMF, at around ambient temperature. Amides of formula (1a) can then be prepared by reaction of the pentafluorophenyl ester with amines of formula (3) in an organic solvent such as a halogenated hydrocarbon, e.g. dichloromethane, at around ambient temperature, optionally in the presence of a tertiary amine base such as triethylamine or diisopropylethylamine. The intermediate acids of formula (2) may be prepared by hydrolysis of esters of formula (5) using a base such as an alkali metal hydroxide, e.g. sodium hydroxide or lithium hydroxide, in water and a solvent such as tetrahydrofuran or an alcohol such as ethanol at a temperature from around ambient to reflux.

Amides of formula (1a) can also be prepared directly from esters of formula (5) by heating with an amine of formula (3) up to the reflux temperature of the amine optionally in the presence of a solvent such as 2-ethoxyethanol either at atmospheric pressure or under pressure in a sealed tube (Scheme 2).

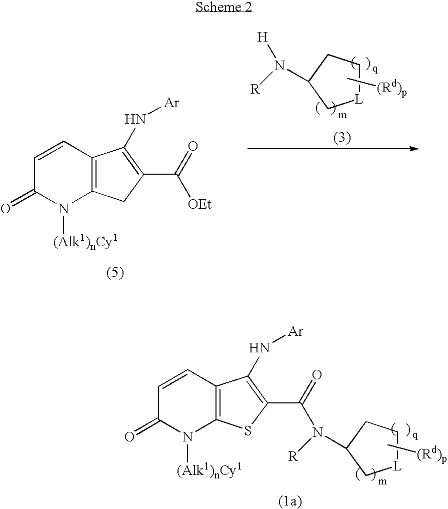

The intermediate esters of formula (5) may be prepared by the methods set out in Scheme 3 below. In the Scheme the preparation of an ethyl ester is specifically shown, but it will be appreciated that other esters may be obtained by simply varying the ester starting material and if appropriate any reaction conditions.

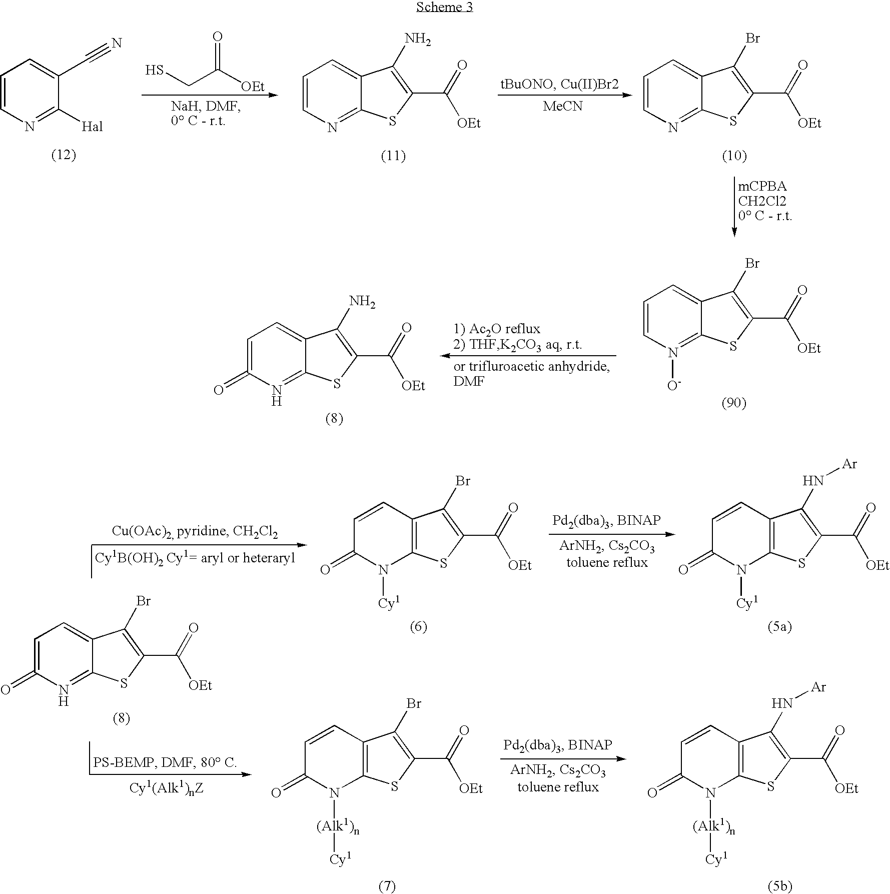

Thus, in Scheme 3 a compound of formula (5a) or (5b) may be prepared by reaction of a compound of formula (6) or (7) with an amine ArNH₂ in the presence of a palladium catalyst. The reaction may be conveniently carried out in a solvent such as toluene at an elevated temperature, e.g. the reflux temperature, using a catalyst such as tris(dibenzylideneacetone)-dipalladium(0), a phosphine ligand such as 2,2′-bis(diphenylphosphino)-1,1′-binaphthyl, and a base such as caesium carbonate. Where desired, alternative reaction conditions may be used, for example as described in the literature [Luker et al., Tetrahedron Leff., 2001, 41, 7731; Buchwald, S. L., J. Org. Chem., 2000, 65, 1144; Hartwig, J. F., Angew. Chem. Int. Ed. Engl., 1998, 37, 2046].

Intermediates of formula (7) may be prepared by reaction of a compound of formula (8) with an alkylating agent of formula Cy¹(Alk¹)ₙZ, where Z is a leaving group such as a halogen atom, e.g. a chlorine, bromine or iodine atom, or a sulphonyloxy group such as an alkylsulphonyloxy, e.g. trifluoromethylsulphonyloxy, or arylsulphonyloxy, e.g. phenylsulphonyloxy, group.

The reaction may be performed in the presence of a solvent, for example a substituted amide such as N,N-dimethylformamide, optionally in the presence of a base, for example an inorganic base such as sodium hydride, or an organic base such as an organic amine, e.g. a cyclic amine such as 1,5-diazabicyclo[4.3.0]non-5-ene, or a resin-bound organic amine such as resin-bound 2-tert-butylimino-2-diethylamino-1,3-dimethyl-perhydro-1,3,2-diazaphosphorine (PS-BEMP), at an elevated temperature, for example 80 to 100° C.

Intermediates of formula (6) may be prepared by the reaction of a compound of formula (8) with a boronic acid of formula Cy¹B(OH)₂ in which Cy¹ is an aryl or heteroaryl group. The reaction may be performed in an organic solvent, for example a halogenated hydrocarbon such as dichloromethane or dichloroethane, in the presence of a copper reagent, for example a copper(I) salt such as CuI or for example a copper (II) reagent such as copper(II) acetate, optionally in the presence of an oxidant, for example 2,2,6,6-tetramethylpiperidine-1-oxide or pyridine-N-oxide, optionally in the presence of a base, for example an organic amine such as an alkylamine, e.g. triethylamine, or an aromatic amine, e.g. pyridine, at a temperature from around ambient to the reflux temperature [see for example Chan, D. T. et al, Tetrahedron Letters, 1998, 2933; Lam, P. Y. S. et al., Tetrahedron Letters, 2001, 3415].

Intermediates of formula (6) where Cy¹ is an aryl or heteroaryl group may also be prepared by nucleophilic aromatic substitution of a suitably activated aryl or heteroaryl halide with a compound of formula (8). The reaction may be performed in a dialkylamide solvent such as DMF in the presence of a base such as a metal hydride, e.g. sodium hydride, at a temperature from around ambient to 100° C. Suitably activated aryl or heteroaryl halides are those with an electron-withdrawing substituent such as a nitro, cyano or ester group, e.g. a chloro- or fluoro-nitrobenzene or 2-chloro-5-nitropyridine. Alternatively a nitrogen-containing heteroaryl halide can be activated to nucleophilic substitution by N-oxidation, e.g. 2-chloropyridine N-oxide.

It will be appreciated that if desired the reactions just described may be carried out in the reverse order so that the amination using ArNH₂ is performed first with the intermediate of formula (8) followed by alkylation/arylation to yield the compound of formula (5). It may be necessary to protect the nitrogen function of compounds of formula (8) during the course of these reactions. Such protection may be achieved by O-alkylation with an alkyl halide, e.g. cyclopropylmethyl bromide, or an arylalkyl bromide, e.g. benzyl bromide, as shown in Scheme 4.

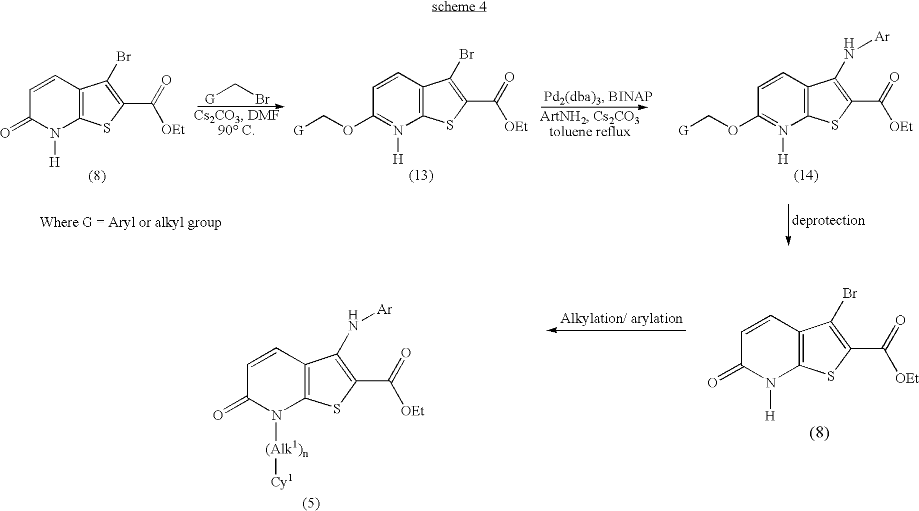

The O-alkylation reaction may be performed in an organic solvent such as DMF in the presence of a base, for example an inorganic base such as Cs₂CO₃ or an organic base such as an amine, e.g. a cyclic amine such as 1,5-diazabicyclo[4.3.0]non-5-ene, at an elevated temperature, e.g. 80 to 100° C., to give a compound of formula (13). Reaction of the protected compound (13) with ArNH₂ under palladium catalysis can then be performed as previously described to give a compound of formula (14). Deprotection can then be achieved by treating a solution of this compound in an alcohol, e.g. MeOH, with a mineral acid such as concentrated HCl at an elevated temperature, e.g. the reflux temperature, to give a compound of formula (15). Alternatively when benzyl protection is employed then this group may be removed reductively by treating a solution of compound (14) in an organic solvent such as EtOH using a palladium or platinum catalyst, e.g. palladium on carbon or PtO₂, under an elevated pressure of hydrogen at a temperature from around ambient to 60° C. Compounds of formula (15) can then undergo alkylation/arylation reactions as previously described to give compounds of formula (5).

Intermediate pyridinones of formula (8) may be prepared from pyridine N-oxides of formula (9) by sequential reaction with an anhydride, for example acetic anhydride, at an elevated temperature, for example the reflux temperature, followed by reaction with an inorganic base, for example a carbonate such as aqueous potassium carbonate, in a solvent such as an ether, for example a cyclic ether, e.g. tetrahydrofuran, at around ambient temperature. Alternatively the reaction may be performed using trifluoroacetic anhydride in N,N-dimethylformamide from 0° C. to ambient temperature conditions [see for example Konno et al., Heterocycles, 1986, 24, 2169].

Pyridine N-oxides of formula (9) may be formed by oxidation of pyridines of formula (10) using an oxidising agent such as hydrogen peroxide in the presence of an acid such as acetic acid, at an elevated temperature, for example around 70° C. to 80° C., or alternatively by reaction with a peracid such as peracetic acid or m-chloroperoxybenzoic acid in a solvent such as a halogenated hydrocarbon, e.g. dichloromethane, or an alcohol, e.g. tert-butanol, at a temperature from the ambient temperature to the reflux temperature.

Intermediate pyridines of formula (10) in Scheme 3 may be obtained by standard methods such as for example by the Sandmeyer reaction. Thus, for example, a bromide of formula (10) may be prepared by treatment of an aryl amine of formula (11) with an alkyl nitrite, for example tert-butyl nitrite, and a copper salt, for example copper(II) bromide, in the presence of a solvent, for example a nitrile such as acetonitrile, at a temperature from about 0° C. to around 65° C.

Amines of formula (11) may be formed from 2-halopyridine-3-carbonitriles of formula (12) by reaction with a reagent such as ethyl 2-mercaptoacetate. The reaction may be performed in the presence of a solvent such as a substituted amide, for example N,N-dimethylformamide, or an ether, e.g. a cyclic ether such as tetrahydrofuran, or an alcohol such as ethanol, in the presence of a base, for example an inorganic base such as sodium carbonate or a hydride, e.g. sodium hydride, or an organic base such as 1,5-diazabicyclo[4.3.0]non-5-ene or a trialkylamine such as triethylamine, at a temperature between about 0° C. and 100° C. The carbonitrile starting materials are readily available or may be obtained from known compounds using standard procedures.

In another process intermediate esters of formula (5a) may be prepared by the reactions set out in Scheme 5. In the Scheme below R²⁰ represents an ester or nitrile and LG represents a leaving group such as a halogen atom, e.g. chlorine or bromine, or a sulfonyloxy group such as an alkylsulfonyloxy group, e.g. trifluoromethylsulfonyloxy, or an arylsulfonyloxy group, e.g. p-toluenesulfonyloxy.

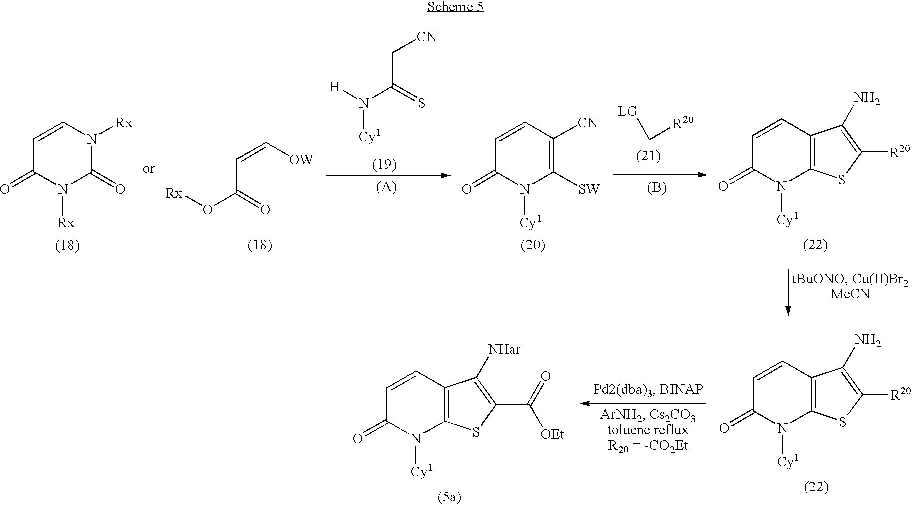

Thus, in step (A) of the reaction scheme a compound of formula (17) or (18), where Rx is an optionally substituted alkyl group, e.g. methyl, and W is a hydrogen atom, metal ion or amine salt, may be reacted with a thioamide of formula (19). The reaction may be performed in the presence of a base. Appropriate bases may include, but are not limited to, lithium bases such as n-butyl- or tert-butyllithium or lithium diisopropylamide (LDA), silazanes, e.g. lithium hexamethyldisilazane (LiHMDS) or sodium hexamethyldisilazane (NaHMDS), carbonates, e.g. potassium carbonate, alkoxides, e.g. sodium ethoxide, sodium methoxide or potassium tert-butoxide, hydroxides, e.g. NaOH, hydrides, e.g. sodium hydride, and organic amines, e.g. triethylamine or N,N-diisopropylethylamine or a cyclic amine such as N-methylmorpholine or pyridine. The reaction may be performed in an organic solvent such as an amide, e.g. a substituted amide such as N,N-dimethylformamide, an ether, e.g. a cyclic ether such as tetrahydrofuran or 1,4-dioxane, an alcohol, e.g. methanol, ethanol or propanol or acetonitrile, at a temperature from ambient to the reflux temperature. In one particular aspect of the process the reaction is achieved using an alkoxide base, especially sodium ethoxide or sodium methoxide, in an alcoholic solvent, especially ethanol, at reflux temperature.

Intermediates of formula (17), where not commercially available, may be prepared using standard methodology. (see, for example, Mir Hedayatullah, J. Heterocyclic Chem., 1981, 18, 339). Similarly, intermediates of formula (18), where not commercially available, may be prepared using standard methodology. For example, they may be prepared in situ by reaction of an acetate, e.g. ethyl acetate, with a base such as sodium methoxide followed by addition of a formate, e.g. methyl formate.

In a similar manner, intermediates of formula (19), if not commercially available, may be prepared using methods known to those skilled in the art (see, for example Adhikari et al., Aust. J. Chem., 1999, 52, 63-67). For example, an isothiocyanate of formula Cy¹NCS may be reacted with acetonitrile in the presence of a base, e.g. NaHMDS, in a suitable solvent, e.g. tetrahydrofuran, optionally at a low temperature, e.g. around −78° C. According to the nature of the group Cy¹, the intermediate of formula (19) may be prepared in situ, for example using the methods as described herein, followed by subsequent addition of a compound of formula (17) or (18).

During the course of this process an intermediate of formula (20) may be formed. If desired the intermediate may be isolated at the end of step (A) and subsequently reacted with intermediate (21) to form the desired amine (22). In some instances, however, it may advantageous not to isolate the intermediate of formula (20) and reaction (B) may be carried out directly with the reaction mixture of step (A).

If a different solvent is used during the second stage of the process, it may be necessary to evaporate the solvent, in vacuo, from the first stage of the process before proceeding with the second stage. Once evaporated, the crude solids from step (A) may be used in the next stage or they may be purified, for example by crystallisation, to yield an isolated intermediate, such as a compound of formula (20).

During step (B) of the process an intermediate of formula (21) may then be added to the reaction mixture or to the crude solids or purified product from step (A) in a suitable solvent. Suitable solvents include, but are not limited to, amides, e.g. a substituted amide such as N,N-dimethylformamide, alcohols, e.g. ethanol, methanol or isopropyl alcohol, ethers, e.g. a cyclic ether such as tetrahydrofuran or 1,4-dioxane, or acetonitrile. The reaction may be performed at a temperature from ambient up to the reflux temperature.

During the course of step (B) an intermediate of formula (24): 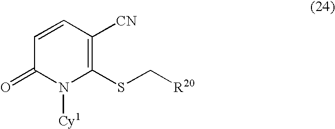
may be observed or even isolated, depending upon the nature of the group R²⁰. The intermediate of formula (24) may be converted to a compound of formula (22) using the methods described above. In this situation it may be necessary to add a base, in order for the reaction to proceed to completion. Appropriate bases include carbonates, e.g. caesium or potassium carbonate, alkoxides, e.g. potassium tert-butoxide, hydrides, e.g. sodium hydride, or organic amines, e.g. triethylamine or diisopropylethylamine or cyclic amines such as N-methylmorpholine or pyridine.

Amines of formula (22) can be converted to bromides of formula (23) by standard methods such as for example by the Sandmeyer reaction as previously described for compounds of formula (11). Compounds of formula (5a) can then be prepared from these bromides by the palladium catalysed amination reactions already described.

It will be appreciated that intermediates of formula (21), where not commercially available, may be prepared using standard methods known to those skilled in the art. For example, alcohol groups may be converted into leaving groups, such as halogen atoms or sulfonyloxy groups, using conditions known to the skilled artisan. For example, an alcohol may be reacted with thionyl chloride in a halogenated hydrocarbon, e.g. dichloromethane, to yield the corresponding chloride. A base, e.g. triethylamine, may also be used in the reaction.

The nitriles of formula (23a), which may be prepared from the reaction scheme depicted in Scheme 5 by providing that R²⁰ is —CN, are useful intermediates in the synthesis of intermediate carboxylic acids of formula (25a). This reaction may be performed by hydrolysis of the nitrile (23a) with a base such as an alkali metal hydroxide, e.g. a 2M aqueous solution of sodium hydroxide in an alcoholic solvent such as methanol or ethanol at reflux.

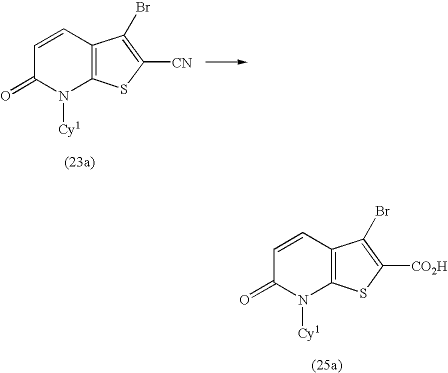

It will be appreciated that intermediates, such as intermediates (17), (18), (19) or (21), if not available commercially, may also be prepared by methods known to those skilled in the art following procedures set forth in references such as Rodd's Chemistry of Carbon Compounds, volumes 1-15 and Supplementals (Elsevier Science Publishers, 1989), Fieser and Fieser's Reagents for Organic Synthesis, volumes 1-19 (John Wiley and Sons, 1999), Comprehensive Heterocyclic Chemistry, ed. Katritzky et al., volumes 1-8, 1984, and volumes 1-11, 1994 (Pergamon), Comprehensive Organic Functional Group Transformations, ed. Katritzky et al., volumes 1-7, 1995 (Pergamon), Comprehensive Organic Synthesis, ed. Trost and Fleming, volumes 1-9 (Pergamon, 1991), Encyclopedia of Reagents for Organic Synthesis, ed. Paquette, volumes 1-8 (John Wiley and Sons, 1995), Larock's Comprehensive Organic Transformations (VCH Publishers Inc., 1989) and March's Advanced Organic Chemistry (John Wiley and Sons, 1992).

In another process amides of formula (1a) may be prepared by the reactions detailed in Scheme 6 below.

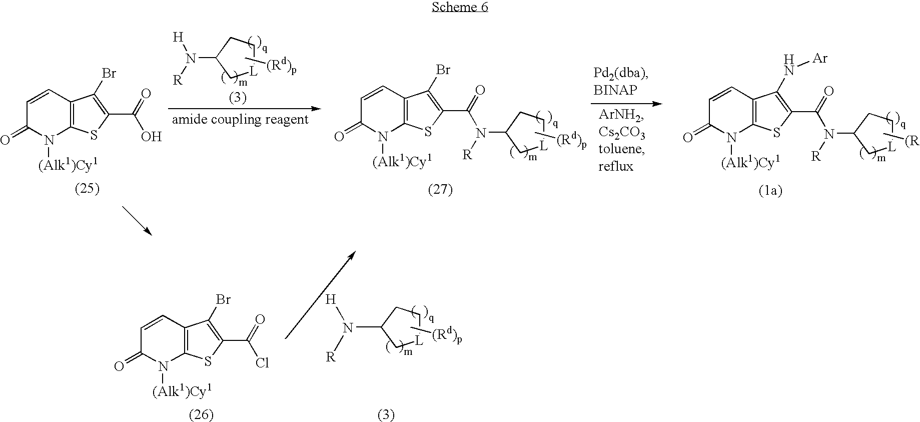

Thus, acids of formula (25) or (25a) may be converted to amides of formula (27) by reaction with amines of formula (3) in the presence of coupling reagents in the same way as previously described for the conversion of compounds (2) to amides of formula (1a). Alternatively the carboxylic acids may be converted to acid chlorides of formula (26) by reaction with a chlorinating agent such as oxalyl chloride optionally in the presence of a catalytic amount of DMF in a solvent such as a halogenated hydrocarbon, e.g. dichloromethane, or an ether, e.g. a cyclic ether such as tetrahydrofuran, at around ambient temperature. The resultant acid chlorides may then be reacted with amines of formula (3) in a solvent such as a halogenated hydrocarbon, e.g. dichloromethane, in the presence of an amine base such as triethylamine at around ambient temperature to give amides of formula (27). Amides of formula (1a) may then be prepared from amides of formula (27) using a palladium-catalysed arylation procedure previously described in Scheme 1. During the course of the reactions described above it may be advantageous or necessary to protect the Rd substituents that may be present. Conventional protecting groups may be used in accordance with standard practice [see, for example, Greene, T. W. in “Protective Groups in Organic Synthesis”, John Wiley and Sons, 1999]. In some instances, deprotection may be the final step in the synthesis of a compound of formula (1a) and the processes according to the invention described hereinafter are to be understood to extend to such removal of protecting groups.

According to a further aspect of the invention a compound of formula (1) in which X is —N(R)— and Y is an —S(O)₂— group may be prepared by the route set out in Scheme 7.

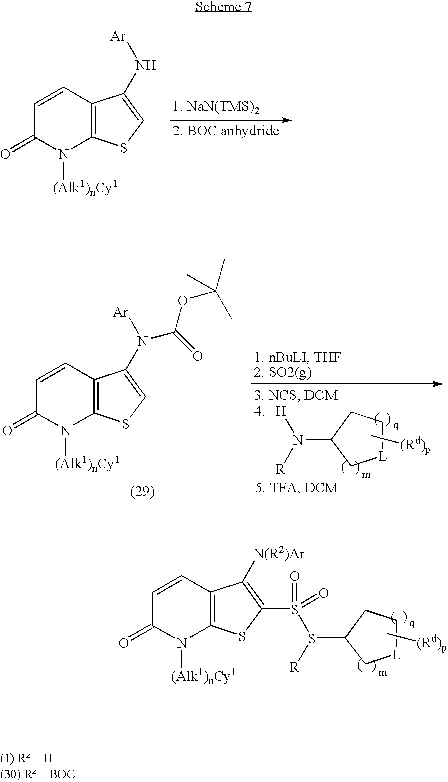

Thus, a compound of formula (29) can be obtained by reaction of a compound of formula (28) with a metal amide base such as sodium bis(trimethylsilyl)amide in a solvent such as an ether, e.g. a cyclic ether such as tetrahydrofuran, at a temperature of around 0° C. and then adding di-tert-butyl dicarbonate in a solvent such as tetrahydrofuran and stirring at ambient temperature. A compound of formula (1) can then be prepared by the following reaction sequence. A compound of formula (29) is treated with a base such as an alkyl lithium, e.g. n-butyllithium, in a solvent such as an ether, e.g. a cyclic ether such as tetrahydrofuran, at a temperature of around −78° C. Sulfur dioxide gas is bubbled through the reaction mixture before allowing the reaction to warm to room temperature. Solvents are removed in vacuo and the crude material dissolved in a solvent such as a halogenated hydrocarbon, e.g. dichloromethane, and the mixture treated with a chlorinating reagent such as N-chlorosuccinimide at around ambient temperature. An amine of formula (3) can then be added to the reaction mixture to produce a compound of formula (30), where R^{z}=tert-butoxycarbonyl. A sulphonamide of formula (1) can then be prepared by treating a compound of formula (30) with an acid, e.g. a mineral acid such as HCl or an organic acid such as trifluoroacetic acid, in a solvent such as a halogenated hydrocarbon, e.g. dichloromethane. Intermediates of formula (28) may be obtained by decarboxylation of compounds of formula (2) with an acid such as a mineral acid, e.g. HCl, in a solvent such as an ether, e.g. a cyclic ether such as tetrahydrofuran or 1,4-dioxane, at a temperature from 50° C. up to the reflux temperature.

A compound of formula (1) in which X is a covalent bond and Y is a —C(O)— group may be prepared by reacting a compound of formula Ar—NH₂ with a compound of formula (27a): 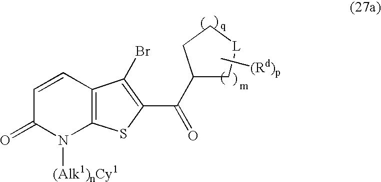
wherein n, m, p, q, R^{d}, L, Alk¹, Cy¹ and Ar are as defined above; in the presence of a palladium catalyst; under conditions analogous to those described above for the conversion of compound (27) to compound (1a).

The intermediates of formula (27a) may be prepared from the corresponding compound of formula (31): 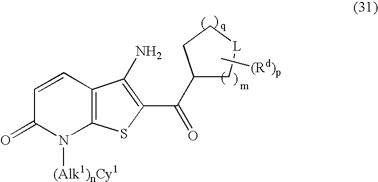
wherein n, m, p, q, R^{d}, L, Alk¹ and Cy¹ are as defined above; by standard methods such as the Sandmeyer reaction as described above for the conversion of compound (11) to compound (10).

The intermediates of formula (31) may be prepared by reacting a compound of formula (20) as defined above with a compound of formula (32): 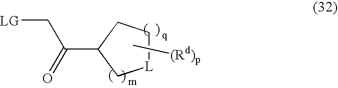
wherein m, p, q, R^{d}, L and LG are as defined above; under conditions analogous to those described above for the reaction between compounds (20) and (21).

Where they are not commercially available, the intermediates of formula (32) may be prepared by methods analogous to those described in the accompanying Examples, or by standard methods known from the art.

Where in the general processes described above intermediates such as alkylating agents of formula Cy¹(Alk¹)ₙZ, reagents of formula HSCH₂CO₂Et and any other intermediates required in the synthesis of compounds of the invention are not available commercially or known in the literature, they may be readily obtained from simpler known compounds by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other intermediates and in particular compounds of formula (1) where appropriate functional groups exist in these compounds. Particular examples of such methods are given in the Examples hereinafter.

Thus for example aromatic halogen substituents in the compounds may be subjected to halogen-metal exchange with a base, for example a lithium base such as n-butyl- or tert-butyllithium, optionally at a low temperature, e.g. around −78° C., in a solvent such as tetrahydrofuran and then quenched with an electrophile to introduce a desired substituent. Thus, for example, a formyl group may be introduced by using N,N-dimethylformamide as the electrophile, a thiomethyl group may be introduced by using dimethyldisulphide as the electrophile, an alcohol group may be introduced by using an aldehyde as the electrophile, and an acid may be introduced by using carbon dioxide as the electrophile. Aromatic acids of formula ArCO₂H may also be generated by quenching Grignard reagents of formula ArMgHal with carbon dioxide.

Aromatic acids of formula ArCO₂H generated by this method and acid-containing compounds in general may be converted to activated derivatives, e.g. acid halides, by reaction with a halogenating agent such as a thionyl halide, e.g. thionyl chloride, a phosphorus trihalide such as phosphorus trichloride, or a phosphorus pentahalide such as phosphorus pentachloride, optionally in an inert solvent such as an aromatic hydrocarbon, e.g. toluene, or a chlorinated hydrocarbon, e.g. dichloromethane, at a temperature from about 0° C. to the reflux temperature, or may be converted into Weinreb amides of formula ArC(O)N(OMe)Me by conversion to the acid halide as just described and subsequent reaction with an amine of formula HN(OMe)Me or a salt thereof, optionally in the presence of a base such as an organic amine, e.g. triethylamine, in an inert solvent such as an aromatic hydrocarbon, e.g. toluene, or a chlorinated hydrocarbon, e.g. dichloromethane, at a temperature from about 0° C. to ambient temperature.

Ester groups such as —CO₂Alk⁶ and —CO₂R⁴ in the compound of formula (1) and intermediates thereto may be converted to the corresponding acid [—CO₂H] by acid- or base-catalysed hydrolysis depending on the nature of the group Alk⁶ or R⁴. Acid- or base-catalysed hydrolysis may be achieved for example by treatment with an organic or inorganic acid, e.g. trifluoroacetic acid, in an organic solvent, e.g. dichloromethane, or a mineral acid such as hydrochloric acid in a solvent such as 1,4-dioxane, or an alkali metal hydroxide, e.g. lithium hydroxide, in an aqueous alcohol, e.g. aqueous methanol.

In a further example, —OR⁶ [where R⁶ represents an alkyl group such as methyl] in compounds of formula (1) and intermediates thereto may be cleaved to the corresponding alcohol —OH by reaction with boron tribromide in a solvent such as a halogenated hydrocarbon, e.g. dichloromethane, at a low temperature, e.g. around −78° C.

Alcohol [—OH] groups may also be obtained by hydrogenation of a corresponding —OCH₂R³¹ group (where R³¹ is an aryl group) using a metal catalyst, for example palladium, on a support such as carbon in a solvent such as ethanol in the presence of ammonium formate, cyclohexadiene or hydrogen, from around ambient to the reflux temperature. In another example, —OH groups may be generated from the corresponding ester [e.g. —CO₂Alk⁶] or aldehyde [—CHO] by reduction, using for example a complex metal hydride such as lithium aluminium hydride or sodium borohydride in a solvent such as methanol.

In another example, alcohol [—OH] groups in the compounds may be converted to a corresponding —OR⁶ group by coupling with a reagent R⁶OH in a solvent such as tetrahydrofuran in the presence of a phosphine, e.g. triphenylphosphine, and an activator such as diethyl, diisopropyl or dimethyl azodicarboxylate.

Aminosulphonylamino [—NHSO₂NHO groups in the compounds may be obtained, in another example, by reaction of a corresponding amine [—NH₂] with sulphamide in the presence of an organic base such as pyridine at an elevated temperature, e.g. the reflux temperature.

In another example, compounds containing a —NHCSR⁷ or —CSNHR⁷ group may be prepared by treating a corresponding compound containing a —NHCOR⁷ or —CONHR⁷ group with a thiation reagent, such as Lawesson's Reagent or P₂S₅, in an anhydrous solvent, for example a cyclic ether such as tetrahydrofuran, at an elevated temperature such as the reflux temperature.

In a further example, amine [—NH₂] groups may be alkylated using a reductive alkylation process employing an aldehyde and a reducing agent. Suitable reducing agents include borohydrides, for example sodium triacetoxyborohyride or sodium cyanoborohydride. The reduction may be carried out in a solvent such as a halogenated hydrocarbon, e.g. dichloromethane, a ketone such as acetone, or an alcohol, e.g. methanol or ethanol, where necessary in the presence of an acid such as acetic acid at around ambient temperature. Alternatively, the amine and aldehyde may be initially reacted in a solvent such as an aromatic hydrocarbon, e.g. toluene, and then subjected to hydrogenation in the presence of a metal catalyst, for example palladium, on a support such as carbon, in a solvent such as an alcohol, e.g. ethanol.

In a further example, amine [—NH₂] groups in compounds of formula (1) and intermediates thereto may be obtained by hydrolysis from a corresponding imide by reaction with hydrazine in a solvent such as an alcohol, e.g. ethanol, at ambient temperature.

In another example, a nitro [—NO₂] group may be reduced to an amine [—NH₂], for example by catalytic hydrogenation using for example hydrogen in the presence of a metal catalyst, for example palladium, on a support such as carbon in a solvent such as an ether, e.g. tetrahydrofuran, or an alcohol, e.g. methanol, or by chemical reduction using for example a metal, e.g. tin or iron, in the presence of an acid such as hydrochloric acid.

In a further example, amine (—CH₂NH₂] groups in compounds of formula (1) and intermediates thereto may be obtained by reduction of nitrites [—CN], for example by catalytic hydrogenation using for example hydrogen in the presence of a metal catalyst, for example palladium on a support such as carbon, or Raney® nickel, in a solvent such as an ether, e.g. a cyclic ether such as tetrahydrofuran, or an alcohol, e.g. methanol or ethanol, optionally in the presence of ammonia solution at a temperature from ambient to the reflux temperature, or by chemical reduction using for example a metal hydride, e.g. lithium aluminium hydride, in a solvent such as an ether, e.g. a cyclic ether such as tetrahydrofuran, at a temperature from 0° C. to the reflux temperature.

In another example, sulphur atoms in the compounds, for example when present in a group L¹ or L², may be oxidised to the corresponding sulphoxide or sulphone using an oxidising agent such as a peroxyacid, e.g. 3-chloroperoxybenzoic acid, in an inert solvent such as a halogenated hydrocarbon, e.g. dichloromethane, at around ambient temperature.

In a further example, N-oxides of compounds of formula (1) may in general be prepared for example by oxidation of the corresponding nitrogen base as described above in relation to the preparation of intermediates of formula (5).

Salts of compounds of formula (1) may be prepared by reaction of compounds of formula (1) with an appropriate base in a suitable solvent or mixture of solvents, e.g. an organic solvent such as an ethe,r e.g. diethyl ether, or an alcohol, e.g. ethanol, using conventional procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt.

In another resolution process a racemate of formula (1) may be separated using chiral High Performance Liquid Chromatography. Alternatively, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode.

Chromatography, recrystallisation and other conventional separation procedures may also be used with intermediates or final products where it is desired to obtain a particular geometric isomer of the invention.

The following Examples illustrate the invention. All temperatures are in ° C. The following abbreviations are used:

NMM—N-methylmorpholine; EtOAc—ethyl acetate;

MeOH—methanol; BOC—tert-butoxycarbonyl;

DCM—dichloromethane; AcOH—acetic acid;

DIPEA—diisopropylethylamine; EtOH—ethanol;

Pyr—pyridine; Ar—aryl;

DMSO—dimethylsulphoxide; iPr—isopropyl;

Et₂O—diethyl ether; Me—methyl;

THF—tetrahydrofuran; h—hour;

MCPBA—3-chloroperoxybenzoicacid; NBS—N-bromosuccinimide;

FMOC—9-fluorenylmethoxycarbonyl; r.t.—room temperature;

DBU—1,8-Diazabicyclo[5,4,0]undec-7-ene;

EDC—1-[3-(dimethylamino)propyl]-3-ethylcarbodiimide hydrochloride;

HOBT—1-hydroxybenzotriazole hydrate;

BINAP—2,2′-bis(diphenylphosphino)-1-1′-binaphthyl;

DMF—N,N-dimethylformamide;

DME—ethylene glycol dimethyl ether;

p.s.i.—pounds per square inch;

MTBE—methyl tert-butyl ether.

All NMRs were obtained either at 300 MHz or 400 MHz.

Compounds were named with the aid of either Beilstein Autonom supplied by MDL Information Systems GmbH, Theodor-Heuss-Allee 108, D-60486 Frankfurt, Germany, or ACD Labs Name (v.6.0) supplied by Advanced Chemical Development, Toronto, Canada.

LCMS retention times (RT) quoted were generated on a Hewleft Packard 1100 LC/MS using the following following method: Phenomenex Luna® 3 μC₁₈(2) 50×4.6 mm column; mobile phase A=0.1% formic acid in water; mobile phase B=0.1% formic acid in MeCN; flow rate of 0.9 mLmin⁻¹, column temperature 40° C.

Gradient:-

| Time (minutes) | % B | % A |
|---|---|---|
| Initial | 5 | 95 |
| 2.0 | 95 | 5 |
| 3.0 | 95 | 5 |
| 5.0 | 5 | 95 |
| 5.5 | end | end |

Where stated alternative LCMS conditions (Conditions B) were used: LCMS retention times (RT) quoted were generated on a Hewlett Packard 1100/ThermoFinnigan LCQ Duo LC/MS system using Electrospray ionisation and the following LC method: Phenomenex Luna® C₁₈(2) 5μ 100 mm×4.6 mm column; mobile phase A=0.08% formic acid in water; mobile phase B=0.08% formic acid in MeCN; flow rate of 3.0 mLmin⁻¹, column temperature 35° C.

Gradient:-

| Time (min) | % A | % B |
|---|---|---|
| 0.00 | 95.0 | 5.0 |
| 4.40 | 5.0 | 95.0 |
| 5.30 | 5.0 | 95.0 |
| 5.32 | 95.0 | 5.0 |
| 6.50 | 95.0 | 5.0 |

Intermediate 1

### Ethyl 3-aminothieno[2,3-b]pyridine-2-carboxylate

A mixture of 2-chlord-3-cyanopyridine (330 g, 2.3 mol), ethyl 2-mercaptoacetate (361.2 g, 3.0 mol), sodium carbonate (265 g, 2.5 mol) and EtOH (1.2L) was heated to reflux for 4.5 hours. The reaction mixture was cooled to ambient temperature and added to water (15L). The resultant precipitate was stirred for 30 minutes and then filtered. The filter cake was washed with two portions of water (2×2.5L) and dried to constant weight under vacuum at 45° C. to yield the title compound as a brown solid (493.1 g, 93.2%). δH (CDCl₃) 8.68 (1H, dd, J 4.7, 1.2 Hz), 7.93 (1H, dd, J 8.5, 1.2 Hz), 7.29 (1H, dd, J 8.5, 4.7 Hz), 5.90 (2H, b), 4.38 (2H, q, J 7.0 Hz), 1.40 (3H, t, J 7.0 Hz). LCMS RT 2.9 minutes, 223 (M+H)⁺.

Intermediate 2

### Ethyl 3-bromothieno[2,3-b]pyridine-2-carboxylate

Intermediate 1 (363.6 g) was added in portions over two hours to a mixture of copper(II) bromide (403.3 g), tert-butyl nitrite (220.6 g) and acetonitrile (3.6L) stirred at a temperature of 20 to 25° C. The mixture was stirred at 20° C. for 2 hours before it was slowly added to 2M HCl(aq) (4.2L). The reaction mixture slurry was filtered and the solids were washed with water (500 mL). The combined filtrate was extracted with ethyl acetate (8L); this ethyl acetate solution was washed with 2M HCl(aq) (2.2L). The solids were dissolved in ethyl acetate (6L); this solution was washed twice with 2M HCl(aq) (4.4L and 2.2L). The two ethyl acetate solutions were then combined and washed with 2M HCl(aq) (2.2L) and twice with water (2×2L). The ethyl acetate solution was then dried (MgSO₄), filtered and concentrated in vacuo at 40 mbar and 60° C. to give a solid residue. This was broken up and dried to constant weight under vacuum at 45° C. to yield the title compound as a brown solid (458.5 g, 97.9%). δH (DMSO-d₆) 8.89 (1H, d, J 4.7 Hz), 8.47 (1H, d, J 8.6 Hz), 7.71 (1H, dd, J 8.6, 4.7 Hz), 4.46 (2H, q, J 7.2 Hz), 1.40 (3H, t, J 7.2 Hz). LCMS RT 3.8 minutes, 288 (M+H)⁺.

Intermediate 3

### Ethyl 3-Bromothieno[2,3-b]pyridine-2-carboxylate N-oxide

To a slurry of Intermediate 2 (214 g, 0.747 mol) in DCM (2140 mL) under nitrogen was added 70% mCPBA (240 g, 0.97 mol) portionwise over 0.5 h. The reaction was then stirred at room temperature for 18 h. The reaction mixture was quenched with water (800 mL) and pH adjusted to 8.5 with 10% w/v sodium carbonate solution (1250 mL). The basic aqueous layer was removed and the organic layer washed with water until pH 7. The organic layer was concentrated in vacuo and the crude title Product was recovered as a tan solid. The crude product was purified by slurrying in MTBE (600 mL) for 1 h at 0-5° C. to give the title compound (174 g, 77%). AH (CDCl₃) 8.44 (1H, dd, J 6.2, 0.8 Hz), 7.87 (1H, dd, J 8.3, 0.8 Hz), 7.48 (1H, dd, J 8.3, 6.2 Hz), 4.49 (2H, q, J 7.1 Hz), 1.48 (3H, t, J 7.1 Hz). LCMS (ES⁺) RT 2.61 minutes, 302 (M+H)⁺.

Intermediate 4

### Ethyl 3-bromo-6-oxo-6,7-dihydrothieno[2,3-b]pyridine-2-carboxylate

To a suspension of Intermediate 3 (95 g, 0.32 mol) in DMF (950 mL) and stirred at room temperature was added trifluoroacetic anhydride (198 g, 131 mL, 0.94 mol) dropwise over a 30 minute period (slight exotherm observed). After complete addition the reaction was stirred for a further 45 minutes at room temperature. The excess trifluoroacetic anhydride was removed under vacuum and the reaction mixture concentrated to approximately half the original volume. The resulting dark-coloured solution was then poured onto a mixture of water (1L) and toluene (400 mL). The mixture was left to stand for around 10 minutes and then the precipitate was collected by filtration. The precipitate was washed with toluene (3×50 mL) and then dried in a vacuum oven at 50-60° C. This gave the title compound as a beige-coloured solid (68.5 g, 72.1%). δH (DMSO-d₆) 12.20 (1H, brs), 7.75 (1H, d, J 9.0 Hz), 6.50 (1H, d, J 9.0 Hz), 4.15 (2H, q, J 7.1 Hz), 1.12 (3H, t, J 7.1 Hz). LCMS (ES⁺) RT 2.86 minutes, 302 ((M+H)⁺, 100%). M.p. 261.7-268.1° C.

Intermediate 5

### Ethyl 3-bromo-6-oxo-7-phenyl-6,7-dihydrothieno[2,3-b]pyridine-2-carboxylate

Method A

A 3L jacketed vessel was charged with Intermediate 4 (100 g, 0.332 mol), CuI (15.8 g, 0.083 mol), phenylboronic acid (80 g, 0.664 mol), pyridine (104 g, 1.32 mol) and acetonitrile (2.0L) and the mixture stirred at 40° C. Compressed air was vigorously blown through the reaction mixture for 6 hours. The compressed air was then turned off and the reaction mixture left to stir at 40° C. overnight. The next day the same process was repeated. After approximately 36 hours, HPLC indicated >97% conversion of starting material to the product. The resulting dark-coloured reaction mixture was poured onto a mixture of water (1.2L) and concentrated hydrochloric acid (300 mL). The mixture was extracted with dichloromethane (2×1.5L) and the combined organics washed with 2M HCl(aq) (2×1.5L). The organic layer was separated, passed through a pad of MgSO₄, and concentrated in vacuo. The crude residue was recrystallised from toluene (600 ml) to give the title compound as a beige solid (93.85 g, 75.0%). δH (CDCl₃) 7.82 (1H, d, J 8.5 Hz), 7.70-7.62 (3H, m), 7.54-7.42 (2H, m), 6.70 (1H, d, J 8.5 Hz), 4.15 (2H, q, J 7.1 Hz), 1.14 (3H, t, J 7.1 Hz). LCMS (ES⁺) RT 3.75 minutes, 378 (M+H)⁺. M.p. 201.6-206.0° C.

Method B (Alternative Procedure)

To a 2 necked round-bottomed flask was added in sequence Intermediate 4 (302 mg, 1.00 mmol), copper(II) acetate (278 mg, 1.50 mmol), phenylboronic acid (488 mg, 4.00 mmol), DCM (5 mL) and pyridine (1 58 mg, 2.00 mmol). The reaction was stirred at room temperature for 18 h with the exclusion of moisture. The reaction was then diluted with DCM (50 mL), washed with 2M HCl(aq) (50 mL), and the aqueous was re-extracted with DCM (50 mL). The combined organics were then washed with water (50 mL), dried (MgSO₄) and concentrated in vacuo. The crude product was purified by a slurry in methanol (12 mL), to give the title compound as a beige solid (270 mg, 72%). δH (CDCl₃) 7.82 (1H, d, J 8.5 Hz), 7.70-7.62 (3H, m), 7.54-7.42 (2H, m), 6.70 (1H, d, J 8.5 Hz), 4.15 (2H, q, J 7.1 Hz), 1.14 (3H, t, J 7.1 Hz). LCMS (ES⁺) RT 3.75 minutes, 378 (M+H)⁺.

Intermediate 6

### Ethyl 3-[(2,4-difluorophenyl)amino]-6-oxo-7-phenyl-6,7-dihydrothieno[2,3-b]pyridine-2-carboxylate

Tris(dibenzylideneacetone)dipalladium(0) (1.21 g, 1.32 mmol) was added to a mixture of Intermediate 5 (10 g, 26.4 mmol), caesium carbonate (12.05 g, 37.0 mmol), 2,4-difluoroaniline (4.1 g, 3.23 mL, 31.7 mmol) and BINAP (1.65 g, 2.64 mmol) in anhydrous toluene (80 mL) and the reaction heated to reflux under nitrogen for 4 days. The reaction was cooled, partitioned between DCM and water and the organic phase dried (MgSO₄) and evaporated in vacuo. The crude residue was triturated with methanol to give the title compound as a white solid (9.87 g). δH (CDCl₃) 8.49 (1H, bs), 7.58-7.40 (3H, m), 7.32-7.25 (2H, m), 7.13-7.04 (1H, m), 7.01 (1H, d, J 9.8 Hz), 6.93-6.86 (1H, m), 6.82-6.75 (1H, m), 6.31 (1H, d, J, 9.8 Hz), 4.20 (2H, q, J 7.1 Hz), 1.23 (3H, J 7.1 Hz). LCMS (ES⁺) RT 4.06 minutes, 427 (M+H)⁺.

Intermediate 7

### Lithium 3-[(2,4-difluorophenyl)amino]-6-oxo-7-phenyl-6,7-dihydrothieno[2,3-b]pyridine-2-carboxylate

A solution of lithium hydroxide monohydrate (686 mg, 16.4 mmol) in water (125 mL) was added to a suspension of Intermediate 6 (6.34 g, 14.9 mmol) in ethanol (250 mL) and THF (125 mL). The reaction was stirred at 85° C. for 4 h before allowing to cool to room temperature. Solvent was removed in vacuo and the residue co-evaporated with toluene (3×50 mL) to give the title compound as a brown solid (6.02 g). δH (DMSO-d₆) 10.04 (1H, bs), 7.81 (3H, m), 7.69 (2H, m), 7.50 (1H, m), 7.48 (1H, d, J 9.6 Hz), 7.16 (2H, m), 7.56 (1H, d, J 9.6 Hz).

Intermediate 8

### Pentafluorophenyl 3-[(2,4-difluorophenyl)amino]-6-oxo-7-phenyl-6,7-dihydrothieno[2,3-b]pyridine-2-carboxylate

1-(3-Dimethylaminopropyl)-3-ethylcarbodiimide (3.42 g, 17.8 mmol) was added to a solution of Intermediate 7 (6.02 g, 14.9 mmol) in DMF (300 mL). The reaction was stirred at room temperature for 30 minutes before adding pentafluorophenol (4.10 g, 22.3 mmol) and then stirred for a further 16 h at r.t. Solvent was removed in vacuo and the residue dissolved in DCM (150 mL), washed with water (2×100 mL), dried (MgSO₄) and concentrated in vacuo. The crude product was purified by column chromatography (silica, 20-40% EtOAc in isohexane) to give the title compound as a white solid (1.71 g). AH (CDCl₃) 8.66 (1H, bs), 7.76 (3H, m), 7.58 (2H, m), 7.47 (1H, m), 7.14 (3H, m), 6.54 (1H, d, J 9.9 Hz). LCMS (ES⁺) RT 4.57 minutes, 565 (M+H)⁺.

Intermediate 9

### Benzyl 3-[((3-[(2,4-difluorophenyl)amino]-6-oxo-7-phenyl-6,7-dihydrothieno[2,3-b]pyridin-2-ylcarbonyl)amino]pyrrolidine-1-carboxylate

Intermediate 8 (300 mg, 0.53 mmol) and benzyl 3-aminopyrrolidine-1-carboxylate (350 mg, 1.6 mmol) in DCM (5 mL) were stirred at r.t. for 18 h. An additional equivalent of benzyl 3-aminopyrrolidine-1-carboxylate (0.53 mmol) was added and the reaction stirred for a further 18 h. The reaction mixture was concentrated in vacuo and purified by column chromatography (silica, 60% EtOAc in isohexane) to give the title compound as a yellow oil (141 mg). LCMS (ES⁺) RT 3.63 minutes, 601 (M+H)⁺.

Intermediate 10

### tert-Butyl(3R)-3-[({3-[(2,4-difluorophenyl)amino]-6-oxo-7-phenyl-6,7-dihydrothieno[2,3-b]pyridin-2-yl}carbonyl)amino]pyrrolidine-1-carboxylate

Intermediate 8 (0.75 g, 1.30 mmol), tert-butyl(3R)-3-aminopyrrolidine-1-carboxylate (272 mg, 1.45 mmol) and triethylamine (1 mL, 7.14 mmol) were dissolved in dichloromethane (20 mL) and stirred at r.t. for 18 h. The reaction mixture was washed with water, dried (sodium sulphate) and was purified by column chromatography (silica, 5% methanol in dichloromethane) to give the title compound as a colourless oil (547 mg). LCMS (ES⁺) RT 3.742 minutes, 566 (M)⁺.

Intermediate 11

### Sodium 3-cyano-6-oxo-1-phenyl-1,6-dihydropyridine-2-thiolate

A solution of sodium methoxide in MeOH (30 wt %, 202.2 g, 1.12 mol) was added to absolute ethanol (360 mL) followed by 1,3-dimethyluracil (75 g, 0.535 mol) and 2-cyano-N-phenylthioacetamide (Adhikari et al., Australian J. Chem., 1999, 52, 63-67) (90 g, 0.511 mol). The resulting mixture was heated at reflux for 8 h and then allowed to cool to ambient temperature overnight. The product was collected by filtration, the filter cake washed with cold ethanol (450 mL) and then dried to constant weight under vacuum at 45° C. to give the title compound as a pale pink solid (130.0 g). The product thus obtained contained residual EtOH and MeOH, estimated at 12.2 wt % by 1H NMR, corresponding to a corrected yield of 114.1 g. δH (DMSO-d₆) 7.32 (2H, m), 7.27-7.18 (1H, m), 7.16 (1H, d, J 9.1 Hz), 6.92 (2H, m), 5.63 (1H, d, J 9.1 Hz). LCMS (Conditions B) (ES⁺) RT 2.43 minutes, 229 (M+H)⁺.

Intermediate 12

### 9H-Fluoren-9-ylmethyl 4-(bromoacetyl)piperidine-1-carboxylate

FMOC-isonipecotic acid (2.0 g, 5.7 mmol) was added to pre-washed sodium hydride (251 mg, 6.3 mmol) in tetrahydrofuran (20 mL). After stirring at room temperature for five minutes then at 60° C. for thirty minutes, thionyl chloride (750 mg, 6.3 mmol) was added causing the precipitated sodium salt to dissolve. After stirring at 60° C. for thirty minutes the reaction was concentrated under reduced pressure then azeotroped with heptane to remove residual thionyl chloride to give the acid chloride 9H-fluoren-9-ylmethyl 4-(chlorocarbonyl)piperidine-1-carboxylate. 1-Methyl-3-nitro-1-nitrosoguanidine (2.94 g, 20 mmol) was added in portions to 40% aqueous potassium hydroxide (30 mL) and diethyl ether (20 mL). The ether layer was decanted, dried over sodium sulphate and added to the acid chloride from above in diethyl ether (20 mL). The reaction was stirred at 0° C. for 2 h and was then treated with 48% hydrogen bromide in acetic acid (5 mL). After stirring at room temperature overnight the reaction mixture was diluted with methanol and concentrated in vacuo. The crude product was purified by column chromatography (silica, 40% DCM in isohexane) to give the title compound (1.24 g). LCMS (ES⁺) RT 4.20 minutes, 450 (M+Na)⁺.

Intermediate 13

### 9H-Fluoren-9-ylmethyl 4-[(3-amino-6-oxo-7-phenyl-6,7-dihydrothieno[2,3-b]pyridin-2-yl)carbonyl]piperidine-1-carboxylate

Intermediate 12 (467 mg, 1.04 mmol), Intermediate 11 (200 mg, 0.8 mmol) and potassium carbonate (221 mg, 1.6 mmol) were stirred in acetonitrile (5 mL) at 50° C. for 4 h. The reaction mixture was cooled, partitioned between dichloromethane and water, the organic phase was dried over sodium sulphate and concentrated in vacuo. The crude product was purified by column chromatography (silica, 0-100% EtOAc in DCM) to give the title compound (212 mg). NMR δH (d6-DMSO) 8.10 (1H, d, J 9.6 Hz), 7.88 (2H, brs), 7.73 (2H, d, J 7.4 Hz), 7.48-7.44 (5H, m), 7.34-7.15 (6H, m), 6.38 (1H, d, J 9.6 Hz), 5.60-4.00 (3H, m), 3.89-3.74 (2H, brm), 2.80-2.65 (2H, brm), 2.51-2.40 (1H, brm), 1.62-1.42 (2H, brm), 1.32-1.22 (2H, brm). LCMS (ES⁺) RT 4.11 minutes, 576 (M+H)⁺.

Intermediate 14

### 9H-Fluoren-9-ylmethyl 4-[(3-bromo-6-oxo-7-phenyl-6,7-dihydrothieno[2,3-b]pyridin-2-yl)carbonyl]piperidine-1-carboxylate

Intermediate 13 (193 mg, 0.34 mmol), tert-butyl nitrite (48.5 mg, 56 mL, 0.47 mmol) and copper(II) bromide (82.5 mg, 0.37 mmol) were mixed in acetonitrile (5 mL) and stirred at 0° C. for 4 h. The solvent was removed in vacuo and the residue partitioned between dichloromethane and water, the organic phase was separated, dried over sodium sulphate and concentrated. The crude product was purified by column chromatography (silica, 0-100% EtOAc in DCM) to give the title compound (166 mg). NMR δH (d6-DMSO) 7.94 (1H, d, J 9.7 Hz), 7.88 (2H, d, J 7.3 Hz), 7.69-7.54 (5H, m), 7.52 (2H, d, J 6.1 Hz), 7.43-7.30 (4H, m), 6.70 (1H, d, J 9.7 Hz), 4.38-4.29 (2H, brm), 4.27-4.24 (1H, m), 4.06-3.75 (2H, brm), 3.56 (1H, brt, J 11.30 Hz), 3.00-2.81 (2H, brm), 1.80-1.75 (2H, brm), 1.35-1.20 (2H, brm). LCMS (ES⁺) RT 5.03 minutes, 641 (M+H)⁺.

Intermediate 15

### Ethyl 3-[(4-fluoro-3-methylphenyl)amino]-6-oxo-7-phenyl-6,7-dihydrothieno[2,3-b]pyridine-2-carboxylate

Intermediate 5 (1.00 g, 2.64 mmol), Pd₂(dba)₃ (0.121 g, 0.132 mmol) and rac-BINAP (0.164 g, 0.264 mmol) were stirred in toluene (12 mL) for 5 min. 4-Fluoro-3-methylaniline (0.397 g, 3.172 mmol) and cesium carbonate (1.205 g, 3.701 mmol) were added and the mixture was heated at reflux under N₂ for 24 h. The mixture was dissolved in THF (100 mL) and washed with water. The combined organics were dried (Na₂SO₄) and concentrated in vacuo. The residue was triturated with MeOH to produce the title compound as a white solid (0.754 g). δH (DMSO-d₆) 8.72 (1H, s), 7.67-7.60 (3H, m), 7.51-7.49 (2H, m), 7.18-7.10 (3H, m), 7.09-6.99 (1H, m), 6.39 (1H, d, J 9.7 Hz), 4.15 (2H, q, J 7.07 Hz), 2.22 (3H, s), 1.72 (3H, t, J 7.08 Hz). LCMS (ES⁺) 423 (M+H)⁺.

Intermediate 16

### Lithium 3-[(4-fluoro-3-methylphenyl)amino]-6-oxo-7-phenyl-6,7-dihydrothieno[2,3-b]pyridine-2-carboxylate

Intermediate 15 (0.494 g, 1.170 mmol) was dissolved in EtOH/THF/H₂O (2:1:1) (20 mL), heated to 80° C. and treated with LiOH.H₂O (0.054 g, 1.287 mmol). Reaction was continued until no starting material remained (as judged by TLC). The solvent was removed in vacuo and the residue azeotroped with toluene to give the title compound as a beige solid (0.284 g). δH (DMSO-d6) 7.81-7.75 (3H, m), 7.64-7.62 (2H, m), 7.41-7.38 (1H, d, J 9.55 Hz), 7.20-7.15 (1H, t, J 9.01 Hz), 7.04-7.03 (1H, br m), 6.93-6.90 (1H, br m), 6.48-6.46 (1H, d, J 9.54 Hz), 2.35 (3H, s). LCMS (ES⁺) 395 (M+H)⁺.

Intermediate 17

### Pentafluorophenyl 3-[(4-fluoro-3-methylphenyl)amino]-6-oxo-7-phenyl-6,7-dihydrothieno[2,3-b]pyridine-2-carboxylate

EDC (0.163 g, 0.852 mmol) was added to a solution of Intermediate 16 (0.284 g, 0.710 mmol) in DMF (10 mL) and the mixture stirred at r.t. for 30 min. Pentafluorophenol (0.196 g, 1.065 mmol) was added and the mixture stirred at r.t. for 24 h. The solvent was removed in vacuo and the residue was dissolved in DCM which was then washed with water, dried (MgSO₄) and concentrated in vacuo. Purification by column chromatography (silica, 50% Hexane/EtOAc) produced the title compound as a white solid (0.226 g). δH (DMSO-d₆) 8.96 (1H, s), 7.07-6.95 (5H, br m), 7.55-7.39 (4H, br m), 6.29 (1H, d, J 9.86 Hz), 2.08 (3H, s). LCMS (ES⁺) 561 (M+H)⁺.

Intermediate 18

### tert-Butyl(3S)-3-[({3-[(2,4-difluorophenyl)amino]-6-oxo-7-phenyl-6,7-dihydrothieno[2,3-b]pyridin-2-yl}carbonyl)amino]pyrrolidine-1-carboxylate

From Intermediate 8 (1 g, 1.77 mmol) and tert-butyl(3S)-3-aminopyrrolidine-1-carboxylate (363 mg, 1.95 mmol) by the method of Intermediate 10 gave the title compound (780 mg, 78%). LCMS (ES⁺) RT 3.766 minutes, 567 (M+H)⁺.

Intermediate 19

### tert-Butyl 4-[({3-[(2,4-difluorophenyl)amino]-6-oxo-7-phenyl-6,7-dihydrothieno[2,3-b]pyridin-2-yl}carbonyl)amino]piperidine-1-carboxylate

Intermediate 8 (515 mg, 0.91 mmol), Et₃N (0.5 mL) and tert-butyl 4-aminopiperidine-1-carboxylate (209 mg, 1.0 mmol) in DCM (15 mL) was stirred at r.t. for 7 days. The reaction was partitioned in DCM/H₂O. Organic phases were washed with aq NaHCO₃ and dried (Na₂SO₄). Purification by chromatography (silica, 2% AcOEt in DCM) gave the title compound as a white solid (337 mg, 64%). δH (DMSO-d₆) 9.18 (1H, s), 7.80 (1H, d, J 7.8 Hz), 7.70-7.60 (3H, m), 7.57-7.52 (2H, m), 7.44-7.35 (2H, m), 7.10-7.0 (2H, m), 6.45 (1H, d, J 9.6 Hz), 3.90-3.80 (3H, m), 2.85-2.70 (2H, m), 1.70-1.54 (2H, m), 1.41 (9H, s), 1.37-1.31 (2H, m). LCMS (ES⁺) RT 3.83 minutes, 581 (M+H)⁺.

Intermediate 20

### Benzyl 4-{[methoxy(methyl)amino]carbonyl}piperidine-1-carboxylate

1-[(Benzyloxy)carbonyl]-4-piperidinecarboxylic acid (1.03 g, 3.91 mmol), EDC (0.9 g, 4.69 mmol), 4-dimethylaminopyridine (0.12 g, 0.98 mmol) and N,O-dimethylhydroxylamine hydrochloride (0.382 g, 3.91 mmol) were dissolved in DCM (50 mL) and treated with triethylamine (2.2 mL, 15.7 mmol). The reaction was stirred at room temperature for 2.5 h then diluted with DCM, washed with 2M HCl aq followed by aq NaHCO₃. The organic phase was dried (Na₂SO₄) and concentrated in vacuo to yield the title compound (1 g). δH (DMSO-d6) 7.40-7.30 (5H, m), 5.08 (2H, s), 4.02 (2H, d, J 13.2 Hz), 3.69 (3H, s), 3.09 (3H, s), 2.94-2.89 (3H, m), 1.83-1.57 (2H, m), 1.48-1.34 (2H, m).

Intermediate 21

### Benzyl 4-acetylpiperidine-1-carboxylate

Intermediate 20 (1.0 g, 3.27 mmol) was dissolved in THF (20 mL) and cooled to 0° C. The reaction was treated with 3M methylmagnesium bromide (1.2 mL, 3.59 mmol) and allowed to warm to r.t. over 1 h. The reaction was quenched with water, extracted into DCM, dried (Na₂SO₄) and concentrated in vacuo. The residue was purified by column chromatography (silica, 0-100% DCM-EtOAc gradient) to give the title compound (0.68 g). δH (CDCl₃) 7.32-7.20 (5H, m), 5.05 (2H, s), 4.10 (2H, br d, J 12.2 Hz), 2.81 (2H, br t, J 11.5 Hz), 2.45-2.35 (1H, m), 2.09 (3H, s), 1.87-1.67 (2H, m), 1.64-1.41 (2H, m).

Intermediate 22

### Benzyl 4-(bromoacetyl)piperidine-1-carboxylate

Intermediate 21 (3.65 g, 14.0 mmol) was dissolved in MeOH (100 mL) and treated at 0° C. with bromine (0.72 mL, 14.0 mmol). After allowing to warm to r.t. over 2 h the solvent was removed in vacuo and the residue redissolved in DCM, washed with aq NaHCO₃, dried (Na₂SO₄) and concentrated. Chromatography (silica, 50% DCM:EtOAc) gave the title compound (4.15 g). δH (CDCl₃) 7.39-7.31 (5H, m), 5.14 (2H, s), 4.24-4.11 (2H, m), 3.96 (2H, m), 3.05-2.89 (2H, m), 2.71 (1H, br t, J 12.6 Hz), 1.92-1.87 (2H, m), 1.70-1.50 (2H, m).

Intermediate 23

### Benzyl 4-[(3-amino-6-oxo-7-phenyl-6,7-dihydrothieno[2,3-b]pyridin-2-yl)carbonyl]piperidine-1-carboxylate

Intermediate 22 (4.15 g, 12.2 mmol), Intermediate 11 (3.05 g, 12.2 mmol) and potassium carbonate (3.37 g, 24.4 mmol) in acetonitrile (100 mL) were heated together at 50° C. for 8 h. The solvent was removed in vacuo and the residue partitioned between DCM and water, the organic phase was separated, dried (Na₂SO₄) and concentrated. Purification by column chromatography (silica, 0-100% DCM-EtOAc gradient) gave the title compound (3.55 g). δH (CDCl₃) 7.68-7.57 (4H, m), 7.43-7.30 (7H, m), 6.80 (2H, br s), 6.63 (1H, d, J 9.6 Hz), 5.14 (2H, s), 4.25-4.18 (2H, br m), 2.85-2.83 (2H, m), 2.60-2.50 (1H, m), 1.85-1.64 (4H, m).

Intermediate 24

### Benzyl 4-[(3-bromo-6-oxo-7-phenyl-6,7-dihydrothieno[2,3-b]pyridin-2-yl)carbonyl]piperidine-1-carboxylate

tert-Butyl nitrite (1.71 mL, 14.4 mmol) was dissolved in acetonitrile (50 mL) and treated with cupric bromide (2.5 g, 11.3 mmol). Intermediate 23 (2.5 g, 11.3 mmol) was added and the reaction stirred for 0.5 h. The reaction was quenched with 2M HCl and extracted into DCM, washed with water, dried (Na₂SO₄) and concentrated. Purification by column chromatography gave the title compound (3.02 g). δH (CDCl₃) 7.83 (1H, d, J 9.7 Hz), 7.64-7.53 (3H, m), 7.39-7.28 (7H, m), 6.74 (1H, d, J 9.7 Hz), 5.13 (2H, s), 4.26-4.21 (2H, m), 3.67-3.58 (1H, m), 2.97 (2H, br t, J 11.7 Hz), 2.00-1.82 (2H, m), 1.77-1.63 (2H, m).

Intermediate 25

### Benzyl 4-({3-[(2,4-difluorophenyl)amino]-6-oxo-7-phenyl-6,7-dihydrothieno[2,3-b]pyridin-2-yl}carbonyl)piperidine-1-carboxylate

BINAP (112 mg, 0.18 mmol) and tris(dibenzylideneacetone)dipalladium(0) (82.4 mg, 0.09 mmol) were dissolved in toluene (20 mL) and degassed for 5 min. Cesium carbonate (828 mg, 2.54 mmol) and Intermediate 24 (1 g, 1.81 mmol) were added and the reaction again degassed. Finally 2,4-difluoroaniline (285 mg, 2.2 mmol) was added and the reaction degassed for a further 5 min. After stirring at 100° C. under nitrogen for 18 h the reaction was cooled, diluted with DCM, washed with water, dried (Na₂SO₄) and concentrated in vacuo. Purification by column chromatography (silica, Et₂O-DCM, 10:1) gave the title compound (620 mg). δH (CDCl₃) 10.45 (1H, s), 7.67-7.57 (3H, m), 7.41-7.27 (8H, m), 6.99 (1H, d, J 9.2 Hz), 6.99-6.91 (2H, m), 6.35 (1H, d, J 9.9 Hz), 5.12 (2H, s), 4.30-4.15 (2H, m), 2.92-2.82 (2H, m), 2.65-2.55 (1H, m), 1.82-1.72 (4H, m).

Intermediate 26

### Benzyl 4-({3-[(6-methylpyridin-2-yl)amino]-6-oxo-7-phenyl-6,7-dihydrothieno[2,3-b]pyridin-2-yl}carbonyl)piperidine-1-carboxylate

From Intermediate 24 (900 mg, 1.63 mmol) and 2-amino-6-methylpyridine (212 mg, 1.96 mmol) by the method of Intermediate 25 to give the title compound (1 g). δH (CDCl₃) 10.95 (1H, s), 7.83 (1H, d, J 9.9 Hz), 7.67-7.52 (4H, m), 7.43-7.26 (7H, m), 6.85 (1H, d, J 7.4 Hz), 6.76 (1H, d, J 8.1 Hz), 6.46 (1H, d, J 9.7 Hz), 5.11 (2H, s), 4.23-4.13 (2H, m), 2.91-2.74 (2H, m), 2.73-2.56 (1H, m), 2.44 (3H, s), 1.83-1.68 (4H, m). LCMS (ES⁺) RT 3.892 minutes, 579 (M+H)⁺.

### EXAMPLE 1

### 3-[(2,4-Difluorophenyl)amino]-N-[(1R*,2S*)-2-hydroxycyclopentyl]-6-oxo-7-phenyl-6,7-dihydrothieno[2,3-b]pyridine-2-carboxamide

To a solution of Intermediate 8 (200 mg, 0.354 mmol) in DCM (4 mL) was added cis-2-aminocyclopentanol hydrochloride (97 mg, 0.709 mmol) and diisopropylethylamine (0.14 mL, 0.78 mmol) and the reaction stirred at r.t. for 18 h. A further equivalent of the aminocyclopentanol (48.5 mg, 0.354 mmol) and diisopropylethylamine (0.07 mL, 0.39 mmol) was added and the reaction stirred for a further 7 h. Solvent was removed in vacuo and the residue purified by column chromatography (silica, 20-60% EtOAc in isohexane) to give the title compound as an off-white solid (115 mg). δH (CDCl₃) 8.75 (1H, s), 7.47-7.55 (3H, m), 7.32 (2H, m), 7.12 (1H, d, J 9.7 Hz), 7.00-6.94 (1H, m), 6.89-6.83 (1H, m), 6.76 (1H, m), 6.36 (1H, d, J 9.7 Hz), 5.94 (1H, d, J 6.7 Hz), 4.09-4.04 (2H, m), 1.98-1.53 (4H, m), 1.51-1.41 (3H, m). LCMS (ES⁺) RT 3.32 minutes, 482 (M+H)⁺.

### EXAMPLE 2

### 3-[(2,4-Difluorophenyl)amino]-N-[(1R*,2R*)-2-hydroxycyclopentyl]-6-oxo-7-phenyl-6,7-dihydrothieno[2,3-b]pyridine-2-carboxamide

To a solution of Intermediate 8 (200 mg, 0.354 mmol) in DCM (4 mL) was added trans-2-aminocyclopentanol (72 mg, 0.709 mmol) and the reaction stirred at r.t. for 18 h. A further 3 equivalents of the aminocyclopentanol (108 mg) were added and the reaction stirred for a further 24 h. Solvent was removed in vacuo and the residue purified by column chromatography (silica, 50-100% EtOAc in isohexane) to give the title compound as a yellow solid (145 mg, 85%). δH (CDCl₃) 8.70 (1H, bs), 7.57-7.51 (3H, m), 7.34 (2H, m), 7.08 (1H, d, J 9.8 Hz), 7.05-6.99 (1H, m), 6.91-6.86 (1H, m), 6.81-6.76 (1H, m), 6.36 (1H, d, J 9.8 Hz), 5.51 (1H, d, J 4.1 Hz), 3.93-3.86 (1H, m), 3.85-3.82 (1H, m), 2.06-2.00 (1H, m), 1.97-1.90 (1H, m), 1.75-1.68 (1H, m), 1.60-1.50 (1H, m), 1.30-1.20 (1H, m). LCMS (ES⁺) RT 3.24 minutes, 482 (M+H)⁺.

### EXAMPLE 3

### 3-[(2,4-Difluorophenyl)amino]-N-[(1S,2S)-2-hydroxycyclopentyl]-6-oxo-7-phenyl-6,7-dihydrothieno[2,3-b]pyridine-2-carboxamide

To a solution of Intermediate 8 (200 mg, 0.35 mmol) in DCM (5 mL) was added (1S,2S)-2-aminocyclopentanol (110 mg, 1.10 mmol) and diisopropylethylamine (198 μL, 1.13 mmol) and the reaction heated in a microwave for 60 minutes (50° C., 100 Watts). The reaction mixture was washed with water and concentrated in vacuo. The crude product was purified by column chromatography (silica, 65% EtOAc in isohexane; and then silica, 5% THF in DCM) to give the title compound as a white solid (57 mg). NMR δH (CDCl₃) 7.55 (3H, m), 7.32 (2H, m), 6.95 (3H, m), 6.68 (1H, m), 6.36 (1H, d, J 9.8 Hz), 5.55 (1H, d, J 4.3 Hz), 3.86 (2H, m), 2.0-1.50 (6H, m). LCMS (ES⁺) RT 3.27 minutes, 482 (M+H)⁺.

### EXAMPLE 4

### 3-[(2,4-Difluorophenyl)amino]-N-[(1R,2R)-2-hydroxycyclopentyl]-6-oxo-7-phenyl-6,7-dihydrothieno[2,3-b]pyridine-2-carboxamide

To a solution of Intermediate 8 (200 mg, 0.35 mmol) in DCM (5 mL) was added (1R,2R)-2-aminocyclopentanol (10 mg, 1.10 mmol) and diisopropylethylamine (198 μL, 1.13 mmol) and the reaction heated in a microwave for 90 minutes (50° C., 100 Watts). The reaction mixture was washed with water, the organic layer separated, dried (MgSO₄) and concentrated in vacuo. The crude product was purified by column chromatography (silica, 60% EtOAc in isohexane) to give the title compound as a white solid (89 mg). NMR δH (CDCl₃) 8.70 (1H, bs), 7.53 (3H, m), 7.33 (2H, m), 7.00-6.60 (4H, m), 6.37 (1H, d, J 9.8 Hz), 5.53 (1H, m), 4.15 (1H, bs), 3.78 (2H, m), 2.12-1.02 (6H, m). LCMS (ES⁺) RT 3.24 minutes, 482 (M+H)⁺.

### EXAMPLE 5

### rac-3-[(2,4-Difluorophenyl)amino]-6-oxo-7-phenyl-N-(pyrrolidinyl-3-yl)-6,7-dihydrothieno[2,3-b]-2-carboxamide

Intermediate 9 (141 mg, 0.24 mmol) was dissolved in MeOH (20 mL) and palladium hydroxide (20 wt % on carbon, ˜10 mg) added. The reaction mixture was degassed with nitrogen and then subjected to an atmosphere of hydrogen (balloon). The reaction was stirred at r.t. for 4 h and then filtered through a pad of Celite®. The filter pad was washed with MeOH and the combined methanol filtrates concentrated in vacuo. The crude product was purified by preparative hplc to give the title compound as an off-white solid (12 mg). NMR δH (CDCl₃) 9.23 (1H, bs), 8.50 (1H, s), 8.20 (1H, m), 7.72 (2H, m), 7.68 (2H, m), 7.46 (2H, m), 7.17 (2H, m), 6.57 (1H, d, J 9.7 Hz), 4.47 (1H, m), 2.75-3.25 (4H, m), 2.11 (1H, m), 1.80 (1H, m). LCMS (ES⁺) RT 2.31 minutes, 467 (M+H)⁺.

### EXAMPLE 6

### 3-[(2,4-Difluorophenyl)amino]-6-oxo-7-phenyl-N-[(3R)-pyrrolidinyl]-6,7-dihydrothieno[2,3-b]-2-carboxamide

Intermediate 10 (540 mg, 0.95 mmol) was dissolved in dichloromethane (10 mL) and treated with trifluoroacetic acid (2 mL). After stirring at room temperature for 30 minutes the reaction mixture was concentrated and azeotroped with heptane to remove residual trifuoroacetic acid. The crude residue was dissolved in dichloromethane, washed with sodium hydrogen carbonate solution and the organic phase separated and concentrated in vacuo. The crude product was purified by column chromatogaphy (reverse phase silica, 60% ethanol:40% water) to give the title compound as a white solid (390 mg). NMR δH (CDCl₃) 8.88 (1H, s), 7.65-7.58 (3H, m), 7.43-7.40 (2H, m), 7.19 (1H, d, J 9.7 Hz), 7.11-7.03 (1H, m), 6.99-6.92 (1H, m), 6.88-6.83 (1H, m), 6.43 (1H, d, J 9.7 Hz), 5.91 (1H, brd, J 7.0 Hz), 4.50-4.48 (1H, m), 3.17-3.10 (1H, m), 3.06-3.02 (1H, m), 2.97-2.89 (1H, m), 2.86-2.81 (1H, m), 2.23-2.19 (1H, m), 1.71-1.65 (1H, m). LCMS (ES⁺) RT 2.318 minutes, 467(M+H)⁺.

### EXAMPLE 7

### 3-Anilino-7-phenyl-2-(piperidin-4-ylcarbonyl)thieno[2,3-b]pyridin-6(7H)-one

2,2′-Bis(diphenylphosphino)-1,1′-binaphthyl (15.6 mg, 0.025 mmol), and tris(dibenzylideneacetone)dipalladium(0) (11.5 mg, 0.0125 mmol) were mixed in toluene (5 mL), degassed and stirred under nitrogen for ten minutes. Intermediate 14 (160 mg, 0.25 mmol) and caesium carbonate (114 mg, 0.35 mmol) were added and the reaction again degassed. Aniline (28 mg, 0.30 mmol) was added and, after degassing, the reaction was heated at 100° C. for 18 h. The reaction mixture was cooled, diluted with dichloromethane and washed with water. The organic phase was separated, dried (sodium sulphate) and concentrated in vacuo. The crude product was purified by column chromatography (silica, 0-100% EtOH in DCM) to give the title compound as a solid (20 mg). NMR δH (d6-DMSO) 10.17 (1H, s), 8.37 (1H, s), 7.69-7.58 (3H, m), 7.53-7.50 (2H, m), 7.42-7.37 (2H, m), 7.23-7.11 (4H, m), 6.37 (1H, d, J 9.8 Hz), 3.10-2.99 (2H, m), 2.82-2.75 (1H, m), 2.63-2.54 (2H, m), 1.67-1.51 (4H, m). LCMS (ES⁺) RT 2.33 minutes, 430 (M+H)⁺.

### EXAMPLE 8

### 3-[(4-Fluoro-3-methylphenyl)amino]-7-phenyl-2-(Piperidin-4-ylcarbonyl)-thieno]2,3-b]pyridin-6(7H)-one

From Intermediate 14 (3.0 g, 4.7 mmol) and 4-fluoro-3-methylaniline (705 mg, 5.63 mmol) by the method of Example 7 to give the title compound (512 mg, 24%). δH (DMSO-d₆) 10.26 (1H, s), 8.35 (1H, s), 7.69-7.58 (3H, m), 7.52-7.49 (2H, m), 7.23-7.08 (3H, m), 7.05 (1H, d, J 9.8 Hz), 6.36 (1H, d, J 9.8 Hz), 3.11-3.06 (2H, m), 2.80-2.67 (3H, m), 2.24 (3H, s), 1.69-1.59 (4H, m). LCMS (ES⁺) RT 2.399 minutes, 462 (M+H)⁺.

### EXAMPLE 9

### N-(Azetidin-3-yl)-3-[(4-fluoro-3-methylphenyl)amino]-6-oxo-7-phenyl-6,7-dihydrothieno[2,3-b]pyridine-2-carboxamide

A mixture of Intermediate 17 (1 g, 1.8 mmol) and 3-aminoazetidin-1-carboxylic acid tert-butyl ester (338 mg, 1.98 mmol) in DCM (10 mL) was stirred at r.t. for 5 days. The reaction mixture was treated with trifluoroacetic acid (2 mL) and stirred at r.t. for a further 24 h. The solvent was removed in vacuo, the residue redissolved in DCM, washed with aq. NaHCO₃, dried (Na₂SO₄) and concentrated in vacuo. Chromatography on silica (DCM-EtOH) gave the title compound (220 mg, 27%). δH (DMSO-d₆) 8.85 (1H, s), 7.66-7.60 (3H, m), 7.52-7.49 (2H, m), 7.20 (1H, d, J 7.97 Hz), 7.13-7.06 (2H, m), 6.95-6.92 (1H, m), 6.40 (1H, d, J 9.7 Hz), 4.30-4.18 (2H, m), 4.08-4.04 (1H, m), 2.65 (2H, br d, J 5.0 Hz), 2.22 (3H, s), 1.82-1.62 (2H, m). LCMS (ES⁺) RT 2.370 minutes, 449 (M+H)⁺.

### EXAMPLE 10

### 3-[(2,4-Difluorophenyl)amino]-6-oxo-7-phenyl-N-[(3S)-pyrrolidinyl-3-yl]-6,7-dihydrothieno[2,3-b]pyridine-2-carboxamide

Intermediate 18 (560 mg, 1.00 mmol) in DCM (10 mL) was treated with trifluoroacetic acid (2 mL) and stirred at r.t. for 2 h. The reaction mixture was concentrated and chromatographed (reverse-phase silica; 60:40 ethanol:water) to give the title compound (342 mg, 73%). δH (DMSO-d₆) 9.23 (1H, s), 8.17 (1H, br s), 7.97 (1H, d, J 6.4 Hz), 7.69-7.57 (3H, m), 7.54-7.51 (2H, m), 7.43-7.35 (1H, m), 7.27 (1H, d, J 9.7 Hz), 7.17-7.01 (2H, m), 6.43 (1H, d, J 9.7 Hz), 4.41-4.35 (1H, m), 3.28-3.04 (3H, m), 2.98-2.93 (1H, m), 2.12-2.00 (1H, m), 1.96-1.73 (1H, m). LCMS (ES⁺) RT 2.289 minutes, 467 (M+H)⁺.

### EXAMPLE 11

### 3-[(2,4-Difluorophenyl)amino]-N-[(3S)-1-methylpyrrolidin-3-yl]-6-oxo-7-phenyl-6,7-dihydrothieno[2,3-b]pyridine-2-carboxamide

Example 10 (200 mg, 0.43 mmol) and paraformaldhyde (343 mg, 11.4 mmol) in MeOH (3 mL) were treated with 4M HCl in 1,4-dioxane (2 drops) followed by sodium cyanoborohydride (33 mg, 0.53 mmol). The reaction was stirred at r.t. for 2 h. After quenching with 2M HCl the reaction was basified with NaOH, extracted into DCM, washed with water then dried (Na₂SO₄) and concentrated in vacuo. Purification by chromatography (silica; EtOAc-MeOH) gave the title compound (135 mg, 65%). δH (DMSO-d₆) 9.04 (1H, s), 7.93 (1H, d, J 7.3 Hz), 7.69-7.57 (3H, m), 7.54-7.50 (2H, m), 7.40-7.32 (2H, m), 7.03-6.95 (2H, m), 6.44 (1H, d, J 9.7 Hz), 4.31-4.24 (1H, m), 2.59-2.40 (2H, m), 2.34-2.26 (1H, m), 2.24-2.18 (4H, m), 2.10-1.92 (1H, m), 1.60-1.45 (1H, m). LCMS (ES⁺) RT 2.299 minutes, 481 (M+H)⁺.

### EXAMPLE 12

### 3-[(2,4-Difluorophenyl)amino]-N-[(3R)-1-methylpyrrolidin-3-yl]-6-oxo-7-phenyl-6,7-dihydrothieno[2,3-b]pyridine-2-carboxamide

From Example 6 (250 mg, 0.354 mmol) by the method of Example 11 gave the title compound as an off-white solid (142 mg, 55%). δH (DMSO-d₆) 9.10 (1H, s), 8.00 (1H, d, J 7.2 Hz), 7.70-7.64 (3H, m), 7.56 (2H, d, J 7.0 Hz), 7.45-7.38 (2H, m), 7.05-7.00 (2H, m), 6.47 (1H, d, J 9.6 Hz), 4.38-4.29 (1H, m), 2.70-2.58 (1H, m), 2.51-2.45 (1H, m), 2.36-2.31 (1H, m), 2.25-2.22 (1H, m), 2.21 (3H, s), 2.11-2.00 (1H, m), 1.60-1.50 (1H, m). LCMS (ES⁺) RT 2.33 minutes, 481.0 (M+H)⁺.

### EXAMPLE 13

### 3-[(2,4-Difluorophenyl)amino]-6-oxo-7-phenyl-N-(piperidin-4-yl)-6,7-dihydrothieno[2,3-b]pyridine-2-carboxamide

Intermediate 19 (337 mg, 0.58 mmol) was dissolved in HCl in 1,4-dioxane (4N) and stirred at r.t. for 18 h. The mixture was concentrated in vacuo and triturated with Et₂O. Purified by chromatography (silica, 10% to 25% MeOH in DCM) gave the title compound as an off-white solid (235 mg, 81%). δH (DMSO-d₆) 9.25 (1H, br s), 8.60-8.20 (2H, m), 8.00 (1H, d, J 7.35 Hz), 7.71-7.56 (5H, m), 7.44-7.34 (2H, m), 7.13-7.06 (2H, m), 6.48 (1H, d, J 7.35 Hz), 4.05-3.90 (1H, m), 3.27-3.18 (2H, m), 3.00-2.92 (2H, m), 1.87-1.84 (2H, m), 1.68-1.60 (2H, m). LCMS (ES⁺) RT 2.28 minutes, 481.0 (M+H)⁺.

### EXAMPLE 14

### 3-[(2,4-Difluorophenyl)amino]-N-(1-methylpiperidin-4-yl)-6-oxo-7-phenyl-6,7-dihydrothieno[2,3-b]pyridine-2-carboxamide

From Example 13 (197 mg, 0.38 mmol) by the method of Example 11 gave the title compound as an off-white solid (160 mg, 85%). δH (DMSO-d₆) 9.12 (1H, s), 7.80 (1H, d, J 7.7 Hz), 7.71-7.62 (3H, m), 7.57-7.54 (2H, m), 7.44-7.38 (2H, m), 7.07-6.98 (2H, m), 6.48 (1H, d, J 9.6 Hz), 3.70-3.55 (1H, m), 2.60-2.45 (2H, m), 2.13 (3H, s), 2.00-1.80 (2H, m), 1.65-1.55 (2H, m), 1.50-1.35 (2H, m). LCMS (ES⁺) RT 2.28 minutes, 495.0 (M+H)⁺.

### EXAMPLE 15

### 3-[(2,4-Difluorophenyl)amino]-7-phenyl-2-(piperidin-4-ylcarbonyl)-thieno[2,3-b]pyridin-6(7H-one

Intermediate 25 (600 mg, 1.00 mmol) was treated with 48% hydrobromic acid in acetic acid (10 mL) and stirred at r.t. for 30 min. The reaction was diluted with water and washed with hexane, the aqueous phase was extracted with DCM and the organic extract dried (Na₂SO₄) and concentrated. The residue was redissolved in DCM and washed with 2M NaOH, then dried (Na₂SO₄) and concentrated in vacuo. Purification by column chromatography (reverse phase silica, 40%-60% ethanol water gradient) gave the title compound (420 mg). δH (CDCl₃) 10.45 (1H, s), 7.67-7.56 (3H, m), 7.44-7.36 (2H, m), 7.31-7.24 (2H, m), 7.10-6.89 (3H, m), 6.35 (1H, d, J 9.9 Hz), 3.18-3.12 (2H, m), 2.69-2.53 (3H, m), 1.82-1.68 (4H, m). LCMS (ES⁺) RT 2.293 minutes, 466 (M+H)⁺.

### EXAMPLE 16

### 3-[(6-Methylpyridin-2-yl)amino]-7-phenyl-2-(piperidin-4-ylcarbonyl)-thieno[2,3-b]pyridin-6(7H)-one

From Intermediate 26 (1 g, 1.81 mmol) by the method of Example 15 to give the title compound (480 mg). AH (CDCl₃) 10.97 (1H, s), 7.84 (1H, d, J 9.9 Hz), 7.68-7.51 (4H, m), 7.44-7.41 (2H, m), 6.84 (1H, d, J 7.4 Hz), 6.76 (1H, d, J 7.1 Hz), 6.46 (1H, d, J 9.9 Hz), 3.16-3.11 (2H, m), 2.87-2.57 (3H, m), 2.44 (3H, s), 1.81-1.61 (4H, m). LCMS (ES⁺) RT2.042 minutes, 445 (M+H)⁺.

### EXAMPLE 17

### 3-[(4-Fluoro-3-methylphenyl)amino]-2-[(1-methylpiperidin-4-yl)carbonyl]-7-phenylthieno[2,3-b]pyridin-6(7H)-one

Example 8 (275 mg, 0.51 mmol) and paraformaldehyde (400 mg, 2.4 mmol) were suspended in MeOH (5 mL) and treated with sodium cyanoborohydride (38.3 mg, 0.61 mmol). After stirring at r.t. for 24 h the reaction was quenched with 2M HCl, basified with aq NaOH and extracted into DCM. The organic phase was dried (Na₂SO₄) and concentrated in vacuo. Chromatography (reverse phase silica, 40%-60% ethanol-water gradient) gave the title compound (175 mg). AH (DMSO-d₆) 10.18 (1H, s), 7.69-7.58 (3H, m), 7.55-7.48 (2H, m), 7.20-7.17 (2H, m), 7.14-7.19 (1H, m), 7.07 (1H, d, J 9.8 Hz), 6.36 (1H, d, J 9.8 Hz), 2.75-2.71 (2H, m), 2.52-2.42 (1H, m), 2.24 (3H, d, J 1.7 Hz), 2.10 (3H, s), 1.87-1.78 (2H, m), 1.65-1.53 (4H, m). LCMS (ES⁺) RT 2.376 minutes, 476 (M+H)⁺.

Preparation of Activated Human p38α MAPK for Inhibitor Assays

Purification of Human p38α MAPK

Human p38α MAPK, incorporating an N-terminal (His)6 tag, was expressed in baculovirus-infected High-Five™ cells (Invitrogen) according to the manufacturer's instructions. The cells were harvested 72 h post-infection and lysed in phosphate buffered saline (PBS) containing 1% (w/v) β-octylglucoside and Complete, EDTA-free™ protease inhibitors (Roche Molecular Biochemicals). The lysate was centrifuged at 35000×g for 30 min at 4° C. and the supernatant applied to a NiNTA™ column (Qiagen). Bound protein was eluted by 150 mM imidazole in PBS (after a wash with 15 mM imidazole in PBS) and directly applied to a HiTrap Q™ column (AP Biotch). Bound protein was eluted using a 20 column volume, 0 to 1 M NaCl gradient. Fractions containing (His)6-p38 MAPK were aliquotted and stored at −70° C. prior to their activation.

Preparation of GST-MKK6EE-Containing Lysates

E. coli (BL21 pLysS) expressing the constitutively activated form of human MKK6 fused with an N-terminal glutathione-S-transferase tag (GST-MKK6EE) were harvested by centrifugation and frozen at −70° C. Cells were lysed by resuspension in 1/10th the culture volume of PBS containing Complete, EDTA-free™ protease inhibitors followed by sonication on ice for 4×15 sec. Cell debris was removed by centrifugation at 35,000×g and the resultant supernatant stored in aliquots at ˜70° C.

Activation of (His)6-p38 MAPK

0.45 mL of purified (His)₆-p38 MAPK was incubated with 50 μL of the GST-MKK6EE-containing lysate for 30 min at 23° C. in the presence of 1 mM β-glycerophosphate, 10 mM MgCl₂ and 9 mM ATP. The extent of activation was monitored by mass spectrometric detection of the doubly-phosphorylated form of (His)6-p38 MAPK, which routinely comprised greater than 90% of the final (His)₆-p38 MAPK preparation. The activated (His)₆-p38 MAPK was then diluted×10 in PBS and repurified using the method described above. The concentration of purified, activated (His)6-p38 MAPK was measured by UV absorbance at 280 nm using A280, 0.1%=1.2 and the preparation stored in aliquots at −70° C. prior to its use in inhibitor assays.

p38 MAPK Inhibition Assays

Inhibition of Phosphorylation of Biotinylated Myelin Basic Protein (MBP)

The inhibition of p38 MAPK catalysed phosphorylation of biotinylated MBP is measured using a DELFIA based format. The assay was performed in a buffer comprising 20 mM HEPES (pH 7.4), 5 mM MgCl₂ and 3 mM DTT. For a typical IC50 determination, biotinylated MBP (2.5 μM) was incubated at room temperature in a streptavidin-coated microtitre plate together with activated gst-p38 MAPK (10 nM) and ATP (1 μM) in the presence of a range of inhibitor concentrations (final concentration of DMSO is 2 percent). After fifteen minutes the reaction was terminated by the addition of EDTA (75 mM). The microtitre plate was then washed with Tris buffered saline (TBS), prior to the addition of 100 μl of anti-phospho MBP antibody (mouse) together with europium-labeled anti-mouse IgG antibody. After one hour at room temperature the plate was again washed in TBS followed by the addition of Enhancement solution (PerkinElmer Wallac). Fluorescence measurements were performed after a further fifteen minutes at room temperature.

IC50 values are determined from the plot of log₁₀ inhibitor concentration (x-axis) versus percentage inhibition of the fluorescence generated by a control sample in the absence of inhibitor (y-axis).

Purification of Human Peripheral Blood Mononuclear Cells

Peripheral blood mononuclear cells (PBMC) were isolated from normal healthy volunteers. Whole blood was taken by venous puncture using heparinised vacutainers (Becton Dickinson), diluted 1 in 4 in RPMI 1640 (Gibco, UK) and centrifuged at 400 g for 35 min over a Ficoll-paque gradient (Amersham-Pharmacia Biotech, UK). Cells at the interface were removed and washed once followed by a low speed spin (250 g) to remove platelets. Cells were then resuspended in DMEM containing 10% FCS, penicillin 100 units ml⁻¹, streptomycin 50 μg ml⁻¹ and glutamine 2 mM (Gibco, UK).

Inhibitor Dilutions

Inhibitor stocks (20 mM) were kept as a frozen solution (−20° C.) in DMSO. Serial dilutions of inhibitors were performed in DMSO as 250-times concentrated stocks. Inhibitors were diluted 1 in 250 into tissue culture media, prewarmed to 37° C. and transferred to plates containing PBMC. PBMC and inhibitors were incubated together for 30 min prior to addition of LPS. Inhibitors used in whole blood assays were prepared according to a different regime. Using the same stock solution serial dilutions of inhibitors were performed in DMSO. Inhibitors were then diluted 1 in 500 straight into whole blood in a volume of 1 μL. Inhibitor was incubated with whole blood for 30 min prior to the addition of LPS.

LPS Stimulation of PBMC

PBMC were resuspended at a density of 2×10⁵ cells/well in flat-bottomed 96-well tissue culture treated plates. After the addition of inhibitor cells were stimulated with an optimal dose of LPS (E. coli strain B5:055, Sigma, at a final concentration of 1 μg ml⁻¹) and incubated at 37° C. in 5% CO₂/95% air for 18 hours. TNF-α levels were measured from cell free supernatants by sandwich ELISA (BioSource #CHC1751).

LPS Stimulation of Whole Blood

Whole blood was taken by venous puncture using heparinised vacutainers (Becton Dickinson), and 500 μl of blood aliquoted into each well of a 24-well tissue culture treated plate. After the addition of inhibitor cells were stimulated with an optimal dose of LPS (E. coli strain B5:055, Sigma, at a final concentration of 1 μg ml⁻¹) and incubated at 37° C. without CO₂ for 18 hours. TNF-α levels were measured from cell free supernatants by sandwich ELISA (BioSource #CHC1751).

Rat LPS Induced TNF Release

Male Lewis rats (180-200 g) are anaesthetised with Isofluor and injected i.v. with LPS* in a volume of 0.5 ml sterile saline. After 90 minutes blood is collected into EDTA tubes for preparation of plasma samples. Plasma is stored at −70° C. prior to assay for TNF-α by commercial ELISA.

Rat CIA

Female Lewis rats (180-200 g) are anaesthetised with Isofluor and immunised i.d. at the base of the tail with 2×100 μl of emulsion containing 4 mg/ml bovine collagen II in 0.01M acetic acid and Freund's Incomplete Adjuvant at a ratio of 1:1. A polyarthritis develops with onset from about 13 days post sensitisation. The disease is mainly confined to the ankles and is quantified by plethysmometry. Results are expressed as change in paw volume over time.

In the p38 MAPK assays described above compounds of the invention have IC₅₀ values of around 1 μM and below. The compounds of the invention are clearly potent inhibitors of p38 MAP kinase, especially p38α MAP kinase.

1. A compound of formula (1): 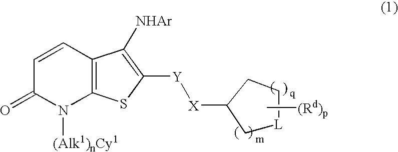
wherein:
X is a covalent bond or the group —N(R)—;
Y is a linking group —C(O)— or —S(O)₂—;
n is zero or the integer 1;
m is the integer 1, 2 or 3;
p is zero or the integer 1, 2, 3 or 4;
q is zero or the integer 1 or 2;
R is a hydrogen atom or a straight or branched C₁₋₆ alkyl group;
R^{d} is —OH, -(Alk²)OH, —OR¹, -(Alk²)OR¹, —NR²R³, -(Alk²)NR²R³ or a straight or branched C₁₋₆ alkyl group;
Alk² is a straight or branched C₁₋₄ alkylene chain;
R¹ is a straight or branched C₁₋₆ alkyl group
R¹ and R³ which may be the same or different, are each independently a hydrogen atom or a straight or branched C₁₋₆ alkyl group;
L is a linking atom or group —O—, —S—, —S(O)—, —S(O₂)—, —CH₂—, —CH(R^{d})—, —C(R^{d})₂— or —NR^{y}—;
R^{y} is a hydrogen atom or a C₁₋₄ alkyl group;
Alk¹ is a straight or branched C₁₋₄ alkylene chain;
Cy¹ is an optionally substituted cycloaliphatic, polycycloaliphatic, heterocycloaliphatic, polyheterocycloaliphatic, aromatic or heteroaromatic group; and
Ar is an optionally substituted aromatic or heteroaromatic group;
or a salt, solvate, hydrate or N-oxide thereof.

2. A compound as claimed in claim 1 wherein Y is —C(O)—.

3. A compound as claimed in claim 1 wherein m is 1 or 2.

4. A compound as claimed in claim 1 wherein q is zero or 1.

5. A compound as claimed in claim 1 wherein L is —CH₂—, —CH(R^{d})—, —NH— or —N(CH₃).

6. A compound as claimed in claim 1 wherein Cy¹ is phenyl, fluorophenyl, chlorophenyl, methylphenyl or cyclopropyl.

7. A compound as claimed in claim 1 wherein Ar is phenyl, difluorophenyl, (chloro)(fluoro)phenyl, (fluoro)(methyl)phenyl, chlorophenyl, cyanophenyl, methylphenyl or methylpyridinyl.

8. A compound as claimed in claim 1 that is
3-[(2,4-Difluorophenyl)amino]-N-[(1R*,2S*)-2-hydroxycyclopentyl]-6-oxo-7-phenyl-6,7-dihydrothieno[2,3-b]pyridine-2-carboxamide;
3-[(2,4-Difluorophenyl)amino]-N-[(1R*,2R*)-2-hydroxycyclopentyl]-6-oxo-7-phenyl-6,7-dihydrothieno[2,3-b]pyridine-2-carboxamide;
3-[(2,4-Difluorophenyl)amino]-N-[(1S,2S)-2-hydroxycyclopentyl]-6-oxo-7-phenyl-6,7-dihydrothieno[2,3-b]pyridine-2-carboxamide;
3-[(2,4-Difluorophenyl)amino]-N-[(1R,2R)-2-hydroxycyclopentyl]-6-oxo-7-phenyl-6,7-dihydrothieno[2,3-b]pyridine-2-carboxamide;
rac-3-[(2,4-Difluorophenyl)amino]-6-oxo-7-phenyl-N-(pyrrolidinyl-3-yl)-6,7-dihydrothieno[2,3-b]-2-carboxamide;
3-[(2,4-Difluorophenyl)amino]-6-oxo-7-phenyl-N-[(3R)-pyrrolidinyl-3-yl]-6,7-dihydrothieno[2,3-b]-2-carboxamide;
3-Anilino-7-phenyl-2-(piperidin-4-ylcarbonyl)thieno[2,3-b]pyridin-6(7H)-one, 3-[(4-Fluoro-3-methylphenyl)amino]-7-phenyl-2-(piperidin-4-ylcarbonyl)-thieno[2,3-b]pyridin-6(7H)-one;
N-(Azetidin-3-yl)-3-[(4-fluoro-3-methylphenyl)amino]-6-oxo-7-phenyl-6,7-dihydrothieno[2,3-b]pyridine-2-carboxamide;
3-[(2,4-Difluorophenyl)amino]-6-oxo-7-phenyl-N-[(3S)-pyrrolidinyl-3-yl]-6,7-dihydrothieno[2,3-b]pyridine-2-carboxamide;
3-[(2,4-Difluorophenyl)amino]-N-[(3S)-1-methylpyrrolidin-3-yl]-6-oxo-7-phenyl-6,7-dihydrothieno[2,3-b]pyridine-2-carboxamide;
3-[(2,4-Difluorophenyl)amino]-N-[(3R)-1-methylpyrrolidin-3-yl]-6-oxo-7-phenyl-6,7-dihydrothieno[2,3-b]pyridine-2-carboxamide;
3-[(2,4-Difluorophenyl)amino]-6-oxo-7-phenyl-N-(piperidin-4-yl)-6,7-dihydrothieno[2,3-b]pyridine-2-carboxamide;
3-[(2,4-Difluorophenyl)amino]-N-(1-methylpiperidin-4-yl)-6-oxo-7-phenyl-6,7-dihydrothieno[2,3-b]pyridine-2-carboxamide,
3-[(2,4-Difluorophenyl)amino]-7-phenyl-2-(piperidin-4-ylcarbonyl)-thieno[2,3-b]pyridin-6(7H)-one;
3-[(6-Methylpyridin-2-yl)amino]-7-phenyl-2-(piperidin-4-ylcarbonyl)-thieno[2,3-b]pyridin-6(7H)-one; or
3-[(4-Fluoro-3-methylphenyl)amino]-2-[(1-methylpiperidin-4-yl)carbonyl]-7-phenylthieno[2,3-b]pyridin-6(7H)-one.

9. A pharmaceutical composition comprising a compound of claim 1, or a pharmaceutically acceptable salt, solvate, hydrate or N-oxide thereof, in association with a pharmaceutically acceptable carrier.

10. (canceled)

11. A method for the treatment or prevention of a disorder for which an inhibitor of p38 MAP kinase is indicated, which comprises administering to a patient in need of such treatment a compound of claim 1, or a pharmaceutically acceptable salt, solvate, hydrate or N-oxide thereof.

12. The method of claim 11 wherein the disorder is an autoimmune disease, inflammatory disease, destructive bone disorder, proliferative disorder, neurodegenerative disorder, viral disease, allergy, infectious disease, heart attack, angiogenic disorder, reperfusion/ischemia in stroke, vascular hyperplasia, organ hypoxia, cardiac hypertrophy, thrombin-induced platelet aggregation, or a condition associated with prostaglandin endoperoxidase synthetase-2 (COX-2).

13. The method of claim 12 wherein the autoimmune disease is rheumatoid arthritis, inflammatory bowel disease, ulcerative colitis, Crohn's disease, multiple sclerosis, diabetes, glomerulonephritis, systemic lupus erythematosus, scleroderma, chronic thyroiditis, Grave's disease, hemolytic anemia, autoimmune gastritis, autoimmune neutropenia, thrombocytopenia, chronic active hepatitis, myasthenia gravis, atopic dermatitis, graft vs host disease, or psoriasis.

14. The method of claim 12 wherein the inflammatory disease is asthma, an allergy, respiratory distress syndrome, or acute or chronic pancreatitis.

15. The method of claim 12 wherein the destructive bone disorder is osteoporosis, osteoarthritis, or multiple myeloma-related bone disorder.

16. The method of claim 12 wherein the proliferative disorder is acute or chronic myelogenous leukemia, Kaposi's sarcoma, metastatic melanoma, or multiple myeloma.

17. The method of claim 12 wherein the neurodegenerative disorder is Parkinson's disease, Alzheimer's disease, cerebral ischemia, or a neurodegenerative disease caused by traumatic injury.

18. The method of claim 12 wherein the viral disease is hepatitis A infection, hepatitis B infection, hepatitis C infection, HIV infection, or CMV retinitis.

19. The method of claim 12 wherein the infectious disease is septic shock, sepsis, or Shigellosis.

20. The method of claim 12 wherein the condition associated with prostaglandin endoperoxidase synthetase-2 (COX-2) is edema, analgesia, neuromuscular pain, headache, dental pain, arthritis pain, or pain caused by cancer.

21. The method of claim 12 wherein the disease or disorder associated with the production of TNF is rheumatoid arthritis, rheumatoid spondylitis, osteoarthritis, gouty arthritis, sepsis, septic shock syndrome, adult respiratory distress syndrome, cerebral malaria, chronic pulmonary inflammatory disease, silicosis, pulmonary sarcoidosis, bone resorption disease, reperfusion injury, graft vs. host reaction, allograft rejections, fever and myalgias due to infection, cachexia secondary to infection, AIDS, ARC or malignancy, keloid formation, scar tissue formation, Crohn's disease, ulcerative colitis, pyresis, HIV, CMV, influenza, herpes.

22. The method of claim 12 wherein the disease or disorder associated with the production of TNF is infection by a veterinary virus selected from equine infectious anemia virus, caprine arthritis virus, visna virus, maedi virus, feline immunodeficiency virus, bovine immunodeficiency virus, and canine immunodeficiency virus.

23. The method of claim 12 wherein the disease or disorder associated with the production of IL-1 is rheumatoid arthritis, osteoarthritis, psoriatic arthritis, traumatic arthritis, rubella arthritis, inflammatory bowel disease, stroke, endotoxemia, toxic shock syndrome, inflammatory reaction induced by endotoxin, diabetes, pancreatic β-cell disease, Alzheimer's disease, tuberculosis, atherosclerosis, muscle degeneration, or cachexia.

24. The method of claim 12 wherein the disease or disorder associated with the production of IL-8 is psoriasis; inflammatory bowel disease; asthma; cardiac, brain, or renal reperfusion injury; adult respiratory distress syndrome; thrombosis; or glomerulonephritis.

25. The method of claim 12 wherein the disease or disorder associated with the production of IL-6 or IL-8 is an infection caused by human rhinovirus (HRV), an enterovirus, a coronavirus, an influenza virus, a parainfluenza virus, a respiratory syncytial virus, or an adenovirus.
